(19) United States
(12) Patent Application Publication
(10) Pub. No.: US 2009/0171394 A1
(43) Pub. Date: Jul. 2, 2009

(54) Devices And Methods For The Treatment Of Facet Joint Disease

(76) Inventor: M. S. Abdou, San Diego, CA (US)

Correspondence Address: M. Samy Abdou, 7855 Entrada Angelica, San Diego, CA, 92127, US

(21) Appl. No.: 12/339,015

(22) Filed: Dec. 18, 2008

Related U.S. Application Data
(60) Provisional application No. 61/008,076, filed on Dec. 18, 2007.
(60) Provisional application No. 61/137,197, filed on Jul. 28, 2008.
(60) Provisional application No. 61/189,341, filed on Aug. 18, 2008.

(51) Int. Cl. A61B 17/70

(52) U.S. Cl. 606/247; 606/279

(57) ABSTRACT

An orthopedic implant is adapted to be implanted within a vertebral facet joint and adapted to maintain motion between adjacent vertebral bodies. An embodiment of the implant includes first segment that rigidly attaches to a facet joint surface of a first vertebra, wherein the first segment contains a cavity that houses a bone forming material which forms a bony fusion with the first vertebra. The implant also includes a second segment having an abutment surface with a facet joint surface of a second vertebra, wherein the second segment does not rigidly attach to the second vertebra.

### REFERENCE TO PRIORITY DOCUMENT

This application claims priority of co-pending U.S. Provisional Patent Application Ser. No. 61/008,076, filed Dec. 18, 2007, U.S. Provisional Patent Application Ser. No. 61/137,197, filed Jul. 28, 2008, and U.S. Provisional Patent Application Ser. No. 61/189,341, filed Aug. 18, 2008. Priority of the aforementioned filing dates is hereby claimed and the disclosure of each Provisional Patent Application is hereby incorporated by reference in its entirety.

### BACKGROUND

The present disclosure relates to treatment of de-stabilizing and degenerative diseases of the posterior spinal elements and, in particular, the facet joint.

A functional spinal unit is made up of two adjacent vertebras bones and the three articulations between them. The two vertebral bones articulate at a single anterior disc space and two posterior facet joints, wherein a single facet joint is located on each side of the sagittal midline. At each lumbar spinal vertebra, for example, one superior articulating process and one inferior articulating process extend from the vertebral bone on each side of the sagittal midline. A surface of the inferior articulating process of a superior vertebra and a surface of the superior articulating process of an inferior vertebra form a single synovial joint, the facet joint, on each side of the sagittal midline and the joint is encased by the joint capsule. Note that each of the superior and inferior articulating processes of a vertebra contains additional surfaces that are not a part of the facet joint. (See Imaging of Vertebral Trauma, 2nd edition (1996)—by Richard A. Daffner; Published by Lippincott-Raven. See Gray's Anatomy: The Anatomical Basis of Medicine and Surgery, 40th edition (2008), Published by Churchill-Livingstone, Elsevier. Each text is hereby incorporated by reference in it's entirety.)

Whether from degenerative disease, traumatic disruption, infection or neoplastic invasion, alteration in the articulation joints between the spinal vertebras can cause significant pain, deformity and disability. Spinal disease is a major health problem in the industrialized world and the surgical treatment of spinal pathology is an evolving discipline. The traditional surgical treatment of abnormal vertebral motion is the complete immobilization and bony fusion of the involved spinal segment and an extensive array of surgical techniques and implantable devices have been formulated to accomplish the treatment objective. More recently, spinal joint repair, replacement and/or distraction have been contemplated as alternative methods in the treatment of pain of spinal origin.

In procedures that attempt to treat spinal disease, it is highly advantageous to utilize a minimally invasive surgical approach that permits access to the diseased segment while minimizing the surgical disruption of the surrounding structures. With these minimally invasive procedures, a percutaneous approach usually provides the least amount of surrounding tissue damage.

Prior attempts at facet joint replacement have involved removal of the entire diseased facet joint or a substantial portion thereof. The removed tissue is replaced with a large prosthesis that fixates into each of the upper and lower vertebral bones that form the joint. Numerous references in the art disclose methods and devices for facet joint repair, replacement and/or fusion. However, the current art continues to have several shortcomings: a) In a first instance, the procedure removes more of the facet joint than is necessary leaving a large defect that must be repaired. In general, the facet joint is an articulation of the inferior articulating surface of the upper vertebra and the superior articulating surface of the lower vertebra. Studies of diseased facet joints have shown that the superior articulating surface of the lower vertebra is usually the diseased segment of the joint. Because of its proximity to the nerve roots, osteophytes and other degenerative outgrowths of the superior articulating surface of the lower vertebra are also the structures that most commonly produce nerve root compression. Removal of the entire joint is unnecessary and the partial removal of the superior articulating surface of the lower vertebra will sufficiently address the diseased segment. b) In a second instance, fixation of the prosthesis onto the underlying bone is often insufficient. In contrast to The large defect caused by the total removal of the facet joint requires repair with a large and substantial prosthesis. The use of a large prosthesis adds to the problem of prosthesis fixation.

### SUMMARY

The present disclosure provides an effective articulation between vertebral bones, wherein the implants are adapted to rigidly attach onto and fuse with at least one of the vertebral bones. All embodiments are adapted for implantation using minimally invasive surgical techniques, while some are specifically adapted for percutaneous implantation under X-ray and/or other imaging techniques.

In an embodiment, a device is implanted within a vertebral facet joint and adapted to maintain motion between adjacent vertebral bodies wherein a first segment of the device is rigidly attached to at least a segment of a facet joint surface of a first vertebra and a second segment of the device forms an abutment surface with at least a segment of a facet joint surface of the second vertebra (or a prosthesis adapted to replace it). Further, the first device segment contains a cavity that is adapted to house a bone forming material and to form a bony fusion with a segment of the first vertebra. The site of bone fusion between the device cavity and the first vertebra may be within the bony segment of a facet joint or outside of the facet joints of the first bone. The second device segment is adapted to abut but not rigidly affix onto or fuse with the second vertebra.

In an embodiment, the device is adapted to be implanted using a percutaneous technique. Resection of the total facet joint, or a substantial portion thereof is not employed. The implanted device serves to limit translation of the first vertebra relative to the second vertebra in the transverse plane and may be also used to reduce the extent of anterior spondylolisthesis between the two adjacent vertebrae. Further, the device may be positioned so that the facet joint surfaces are distracted away from one another and the functional spinal unit (FSU) is placed into slight anterior flexion. This vertebral re-alignment would limit extension and enlarge the cross-sectional area of the spinal canal.

In an other embodiment, a device is adapted to at least partially replace the superior articulating process of the inferior vertebra and maintain motion between an adjacent superior and inferior vertebral bones. A first segment of the device is rigidly attached to at least a segment of a the inferior vertebra and a second segment of the device forms an abutment surface with at least a segment of an inferior articulating process of the superior vertebra (or a prosthesis adapted to replace it). Further, the first device segment contains a cavity that is adapted to house a bone forming material and to form a bony fusion with a bony segment of the inferior vertebra. The second device segment is adapted to abut but not rigidly affix onto or fuse with at least a portion the inferior articulating process of the superior vertebra or with a prosthesis adapted to replace at least a portion of that segment of the superior vertebra.

In an other embodiment, a device is adapted to at least partially replace the inferior articulating process of the superior vertebra and maintain motion between an adjacent superior and inferior vertebral bones. A first segment of the device is rigidly attached to at least a segment of a the superior vertebra and a second segment of the device forms an abutment surface with at least a segment of a superior articulating process of the inferior vertebra (or a prosthesis adapted to replace it). Further, the first device segment contains a cavity that is adapted to house a bone forming material and to form a bony fusion with a bony segment of the superior vertebra. The second device segment is adapted to abut but not rigidly affix onto or fuse with at least a portion the superior articulating process of the inferior vertebra or with a prosthesis adapted to replace at least a portion of that segment of the inferior vertebra.

These implanted devices serve to limit translation of the superior vertebra relative to the inferior vertebra in the transverse plane and may be also used to reduce the extent of anterior spondylolisthesis between the two adjacent vertebrae. Further, the devices may be positioned so that the functional spinal unit (FSU) is placed into slight anterior flexion. This vertebral re-alignment would limit extension and enlarge the cross-sectional area of the spinal canal.

In another embodiment, a device is adapted to at least partially replace a portion of a lamina and both of the ipsilateral inferior and superior articulating processes of the middle vertebra of an assembly of three consecutive vertebral bones. A first segment of the device is rigidly attached to at least a portion of the residual ipsilateral pedicel of the middle vertebra, while a second segment of the device forms an abutment surface with at least a segment of a superior articulating process of the inferior vertebra (or a prosthesis adapted to replace it) and a third segment of the device forms an abutment surface with at least a segment of an inferior articulating process of the superior vertebra (or a prosthesis adapted to replace it). Further, the first device segment contains a cavity that is adapted to house a bone forming material and to form a bony fusion with at least a portion of the residual ipsilateral pedicel of the middle vertebra. The second device segment is adapted to abut but not rigidly affix onto or fuse with at least a segment of a superior articulating process of the inferior vertebra while the third device segment is adapted to abut but not rigidly affix onto or fuse with at least a segment of an inferior articulating process of the superior vertebra. Alternatively, either second or third segments may be adapted to affix onto and fuse with at least a segment of the complimentary articulating process of the adjacent vertebra. In this way, the construct of the three consecutive vertebrae would include a first pair of adjacent vertebral bones that are fused and immobile relative to one another and a second pair of adjacent vertebral bones that are mobile relative to one another.

In another embodiment, the device contains at least one cavity adapted to contain a bone graft material that fuses with the spinous process and/or lamina of superior vertebral bone. The device further contains an abutment surface that is adapted to abut the superior and/or posterior aspects of the superior articulation process of the lower vertebral bone, wherein, preferably, the joint capsule of the facet joint remains substantially intact. In an alternative embodiment, the abutment surface is adapted to abut the posterior aspect of the lamina and/or posterior aspect of the inferior articulation process of the inferior vertebra.

In an additional embodiment, the device contains at least one cavity adapted to contain a bone graft material that fuses with the pedicle portion of superior vertebral bone. The device further contains an abutment surface that is adapted to abut the superior and/or posterior aspects of the superior articulation process of the lower vertebral bone, wherein, preferably, the joint capsule of the facet joint remains substantially intact. This embodiment is also particularly adapted for percutaneous implantation and the method of implantation is also disclosed. In an additional embodiment, a first end of an additional member is connected to the abutment surface of the device that is in contact with the superior articulation process of the lower vertebral bone. A second end of the additional member is positioned immediately inferior to the lower surface of the inferior articulating process of the vertebral bone immediately above the superior vertebral bone. In this way, the device is rigidly anchored to and fused with the superior vertebral bone while providing a limitation of extension between the vertebral bone immediately inferior and the vertebral bone immediately superior to the superior vertebral bone.

These embodiments serve to limit translation of the superior vertebra relative to the inferior vertebra in the transverse plane and may be also used to reduce the extent of anterior spondylolisthesis between the two adjacent vertebrae. Further, the devices may be positioned so that the functional spinal unit (FSU) is placed into slight anterior flexion. This vertebral re-alignment would limit extension and enlarge the cross-sectional area of the spinal canal.

Other features and advantages should be apparent from the following description of various embodiments, which illustrate, by way of example, the principles of the disclosed devices and methods.

### BRIEF DESCRIPTION OF THE DRAWINGS

FIG. 1A illustrates a view of the posterior aspect of the cervical spine

FIG. 1B shows the spine in a lateral view.

FIG. 2 shows a schematic representation of a single facet joint 105

FIG. 3 shows a needle having a distal region that is percutaneously placed into facet joint such as under X-ray imaging.

FIG. 4 shows an instrument having with a handle and an inner cannula sized and shaped to be placed over the needle.

FIG. 5 shows multiple views of the instrument of FIG. 4.

FIG. 6 shows an enlarged view of the anterior aspect of the instrument.

FIG. 7 shows multiple views of inner cannula.

FIG. 8 shows an enlarged view the anterior aspect of the instrument with the inner cannula in place inside the instrument.

FIG. 9 shows the instrument and inner cannula positioned at the joint.

FIG. 10 shows the instrument with the inner cannula and needle removed.

FIG. 11 shows the Instrument removed from joint so that the bone holes are illustrated.

FIG. 12 shows the instrument attached to the facet joint as in actual use.

FIG. 13 shows the implant with the instrument removed.

FIG. 14 shows multiple views of the implant.

FIG. 15 shows an alternate embodiment of the implant.

FIG. 16A illustrates a device embodiment wherein an implant is placed into one vertebral body adjacent to the facet joint.

FIG. 16B shows the implant of FIG. 16A in an implanted state.

FIG. 17 shows an additional embodiment of the implant.

FIG. 18 shows an alternative embodiment of an implant.

FIG. 19 shows the implant of FIG. 18 in an implanted state.

FIG. 20 shows an additional embodiment of the implant.

FIG. 21 shows various views of an alternate embodiment of the implant.

FIG. 22 shows how the implant of FIG. 21 is implanted between the facet joints.

FIG. 23 shows a schematic representation of a cross section of the neck.

FIGS. 24-26 illustrate a method for the selective removal of the superior articulating surface of the lower vertebra and its subsequent replacement with a partial joint prosthesis.

FIGS. 27A and 27B show various views of an exemplary replacement prosthesis.

FIG. 28A illustrates a spinal segment prior to distraction.

FIG. 28B shows the distracted spinal segment after removal of the diseased superior articulating surface of the lower vertebra.

FIG. 29A shows the distracted spinal segment with the superior vertebra removed in order to show the cut surface of the superior articulating surface.

FIG. 29B shows the prosthesis in an implanted state.

FIG. 30 shows the spine after placement of the prosthesis and removal of the distractor.

FIG. 31 shown an alternative embodiment of an implant.

FIG. 32 shows the prosthesis of FIG. 31 attached to bone.

FIG. 33 shows

FIG. 34 shows an exploded view of the device of FIG. 33.

FIGS. 35A and 35B illustrate multiple perspective views of the member of the device of FIG. 33.

FIGS. 36A and 36B show multiple views of an abutment member of the device of FIG. 33.

FIG. 37 shows the device of FIG. 33 in an implanted state.

FIGS. 38A and 38B show the device of FIG. 33 in an implanted state.

FIG. 39A illustrates an intact spinal segment.

FIG. 39B illustrates a segment of bone removed from the lamina and medial articulating surface of the upper vertebral bone.

FIG. 40 shows an additional device embodiment in an assembled state.

FIG. 41 shows the device of FIG. 40 in an exploded state

FIG. 42 shows the device of FIG. 40 attached to a spinal model.

FIGS. 43A-43C show an embodiment of another device.

FIGS. 44-47 show method for the percutaneous implantation of the device of FIGS. 43A-43C under X-ray guidance.

### DETAILED DESCRIPTION

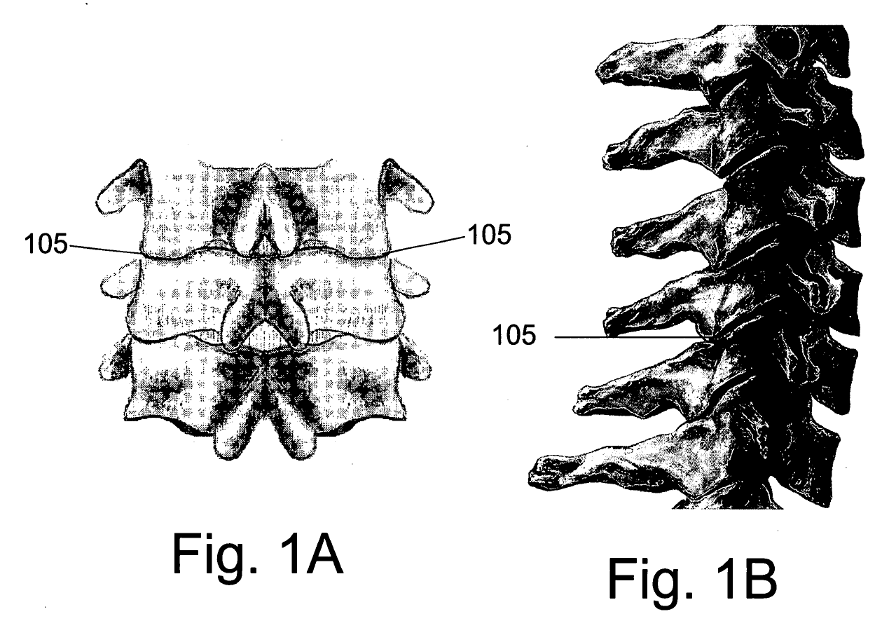

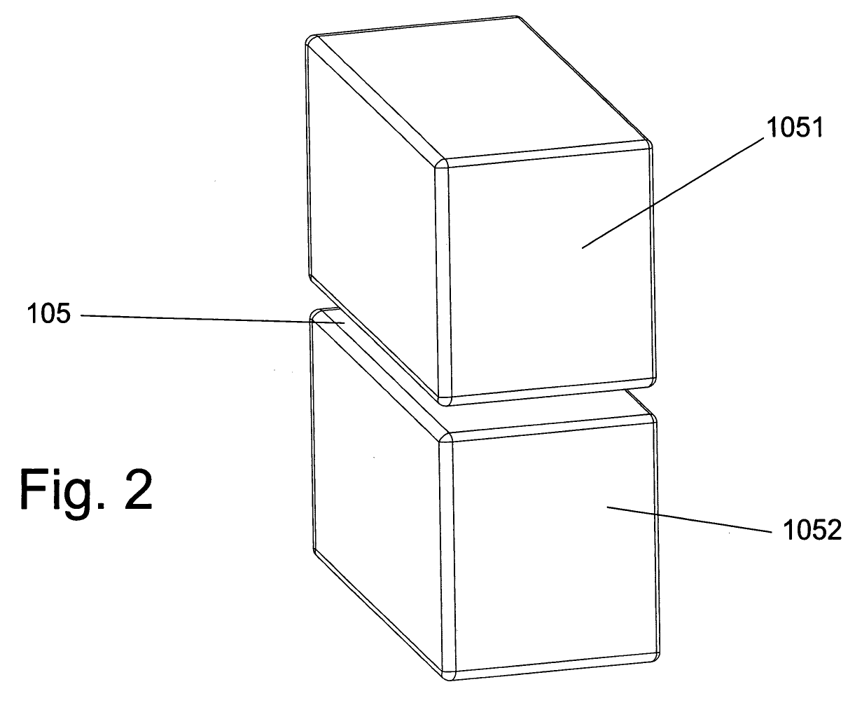

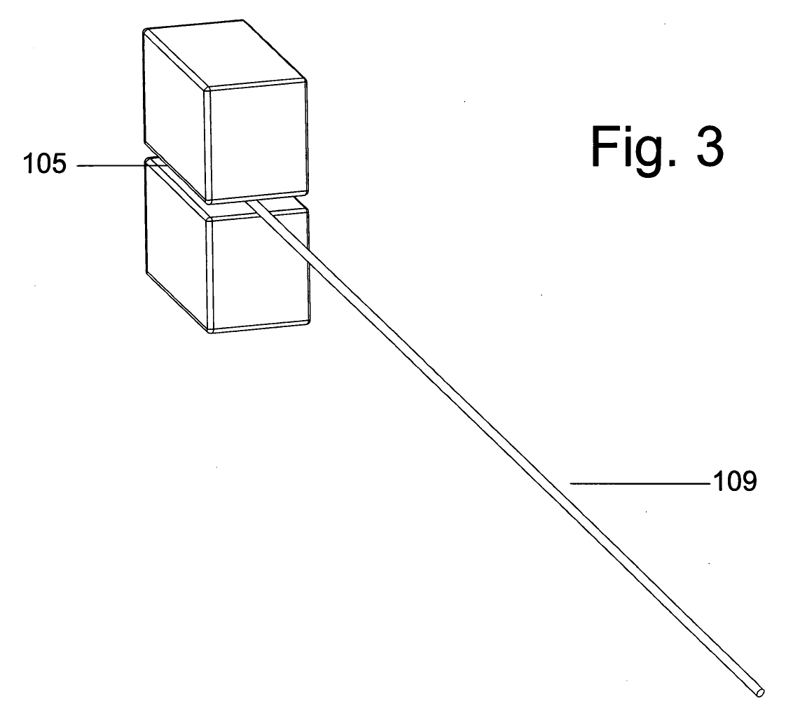

FIG. 1A illustrates a view of the posterior aspect of the cervical spine while FIG. 1B shows the spine in a lateral view. Each functional spinal unit (FSU) of the spine consists of two vertebras that articulate at a single anterior disc space and two posterior facet joints 105. FIG. 2 shows a schematic representation of a single facet joint 105, which is located on one side of the spinal midline. The facet joint 105 is comprised of an upper articulation surface 1052 of a lower vertebra and a lower articulating surface 1051 of an upper vertebra, wherein the articulation surfaces are collectively enclosed within a joint capsule. The facet joint 105 is represented schematically and those skilled in the art will appreciate that actual facet joints may include anatomical details that may differ from those shown in these FIG. 2.

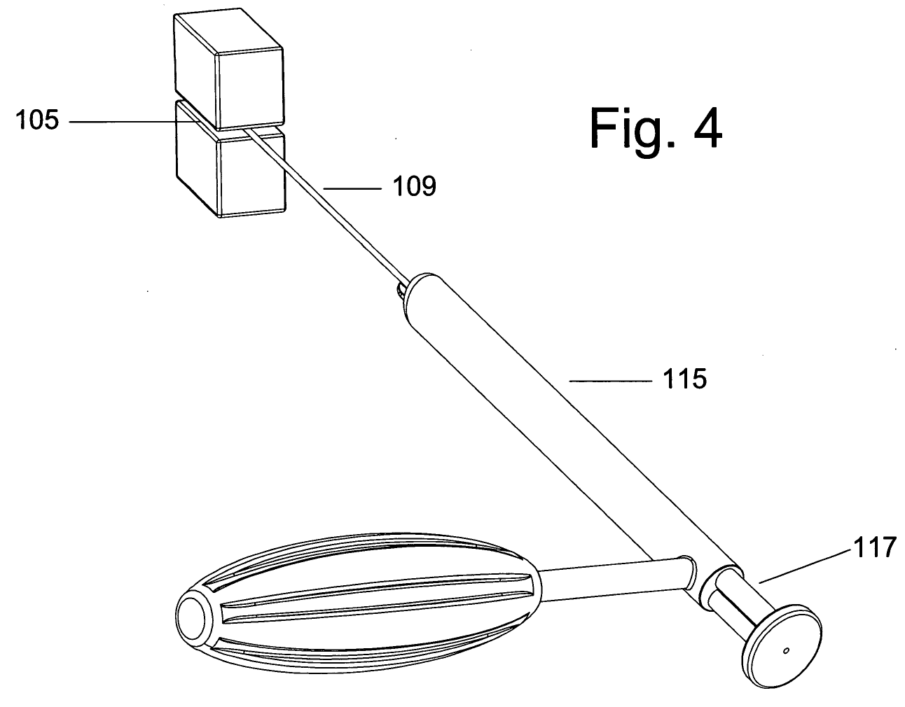

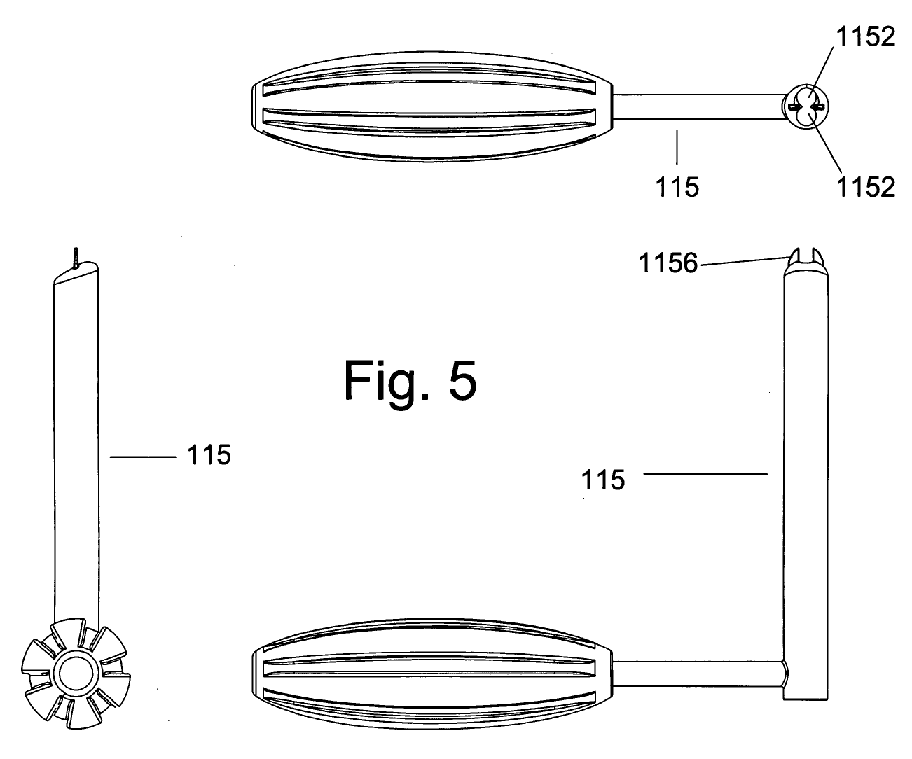

While the disclosed device and method for implantation will be illustrated in the cervical spine, it is understood that they may be alternatively used at any spinal level. The implantation may be performed in a percutaneous manner and guided by X-ray or other imaging techniques. However, it may be alternatively performed under direct visualization using open surgical technique. FIG. 3 shows a needle 109 comprised of an elongate member having a distal region that is percutaneously placed into facet joint 105 under X-ray imaging. FIG. 4 shows an instrument 115 comprised of an elongate member having a handle and an inner cannula 117 sized and shaped to be placed over the needle 109. The cannula 117 is passed over needle 109 such that a distal region of the instrument 115 is seated into joint 105.

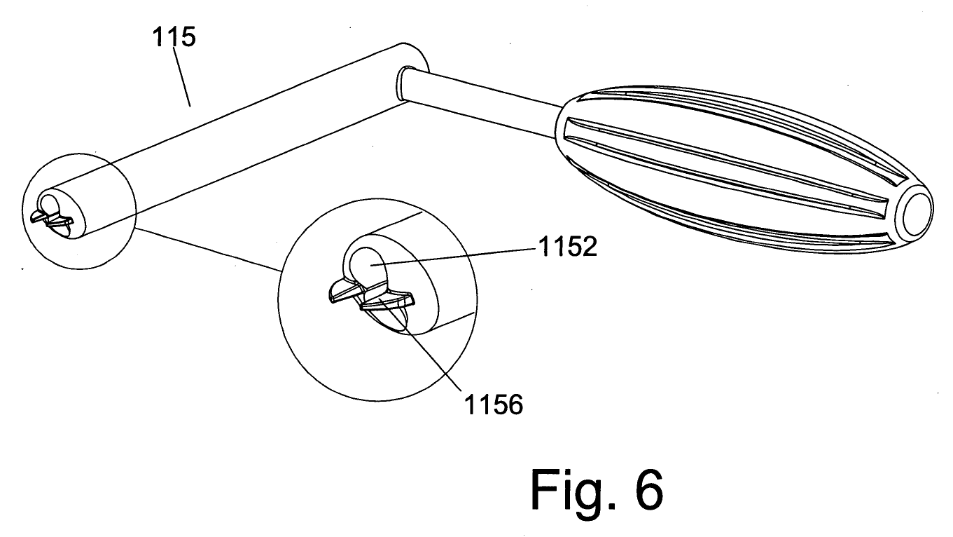

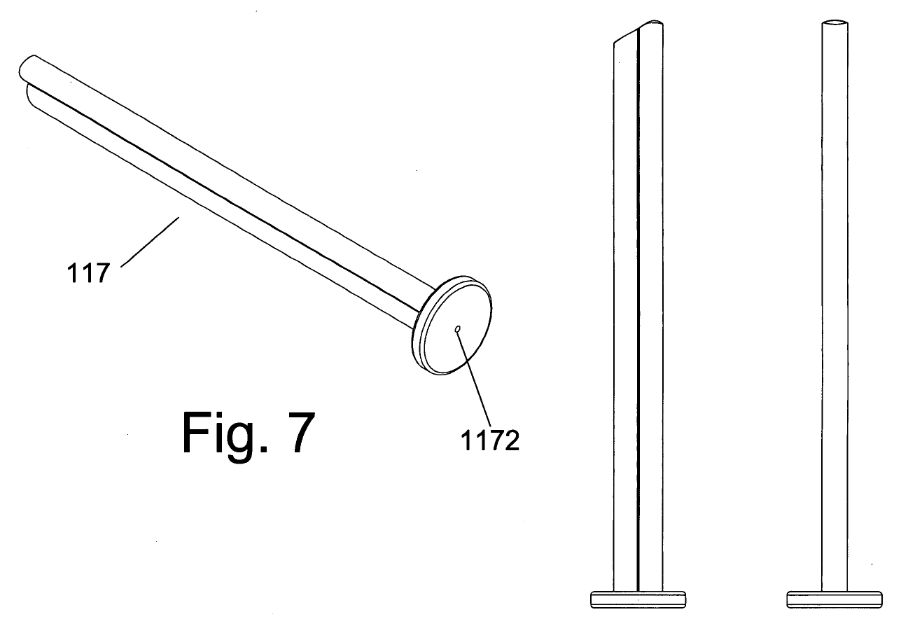

Multiple views of instrument 115 are shown on FIG. 5. The instrument 115 includes a handle that can be grasped by a user. The handle extends laterally from an elongate axis of the main body of the instrument 115 although the handle can have other orientations. The main body includes a pair of internal, overlapping bores 1152 that extend the length of the main body. Each of bores 1152 is a cylindrical cut-out adapted to function as a drill guide. The anterior aspect of the instrument 115 may include one or more protrusions 1156. The protrusions 1156 are sized and shaped to be inserted into the joint and retained therein. FIG. 6 shows an enlarged view of the anterior aspect of the instrument 115. The inner cannula 117 may be rigid or flexible and it is adapted to be positioned within the bores 1152 of the instrument 115

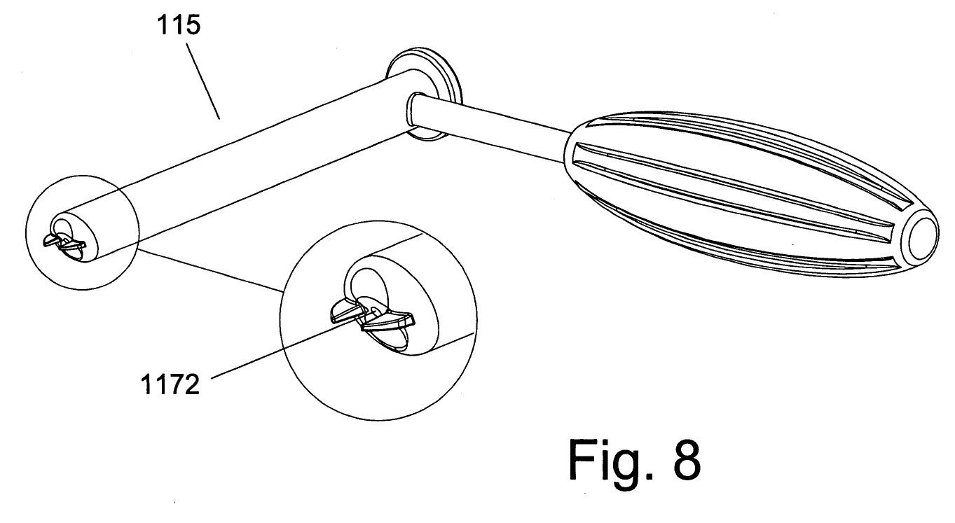

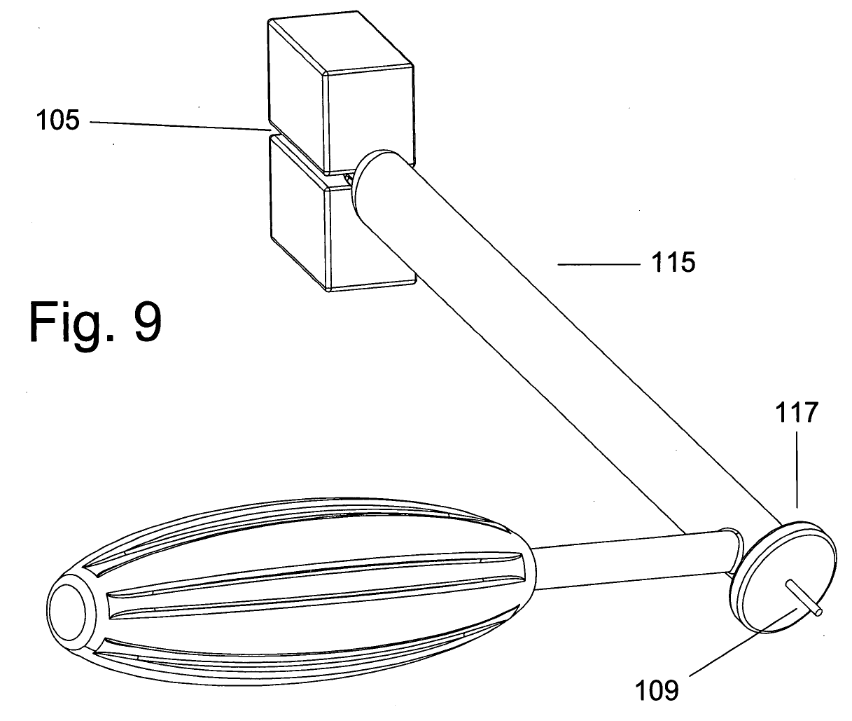

FIG. 7 shows multiple views of inner cannula 117. The inner cannula 117 has a size and shape that complements the size and shape of the bores 1152 of the instrument. Accordingly, the inner cannula 117 can be slidably inserted into the bores 1152, as shown in FIGS. 4 and 8. FIG. 8 shows an enlarged view the anterior aspect of the instrument 115 with the inner cannula 117 in place inside the instrument 115. Note that the cannula 117 contains a central bore 1172 adapted to slidably accept the needle 109. The central bore extends entirely through the cannula 117 such that the bore forms openings in both ends of the cannula for receipt of the needle 109.

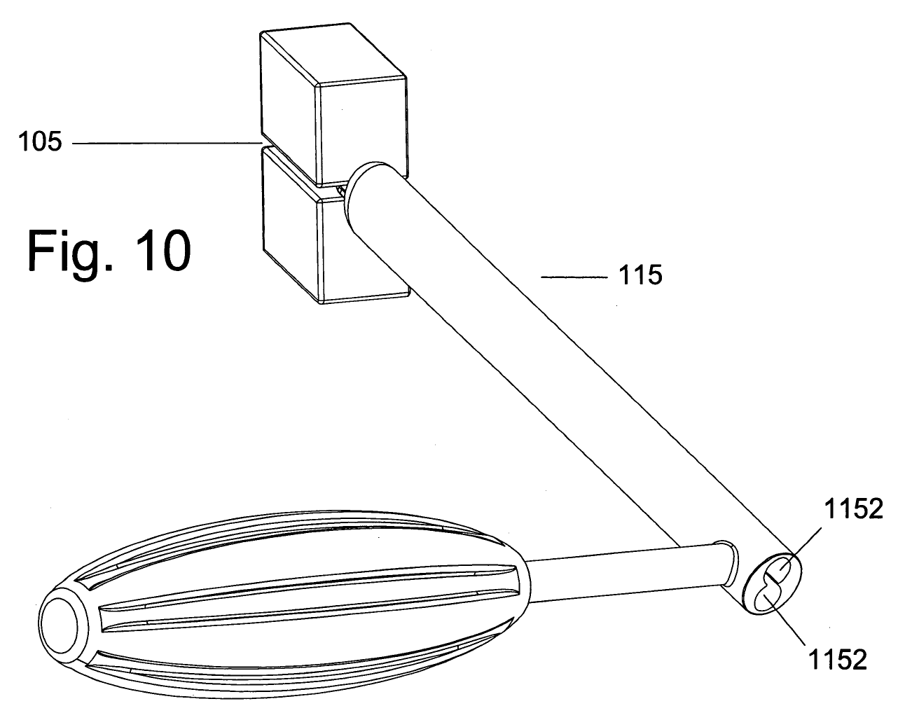

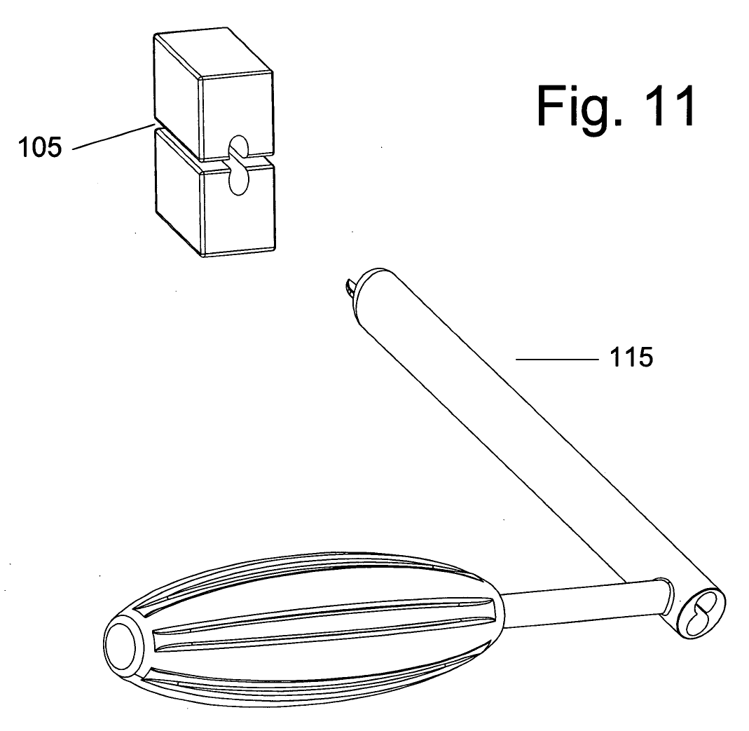

FIG. 9 shows the instrument 115 and inner cannula 117 positioned at the joint 105. Distal regions of the instrument and the cannula 117 are positioned in the joint 105. The needle 109 is also positioned inside the central bore 1172 of the cannula 117. As shown in FIG. 10, the inner cannula 117 and needle 109 are removed from the instrument 115. With the cannula removed, the bores 1152 of instrument 115 are unoccupied. The bores 1152 provide access to the joint 105. A drill bit (not shown) is guided through each of bores 1152 and advanced into the underlying bone. In this way, the drill bits are used to make two holes in the underlying bone. A first hole is placed into the bone of the upper facet joint and a second hole is placed into the bone of the lower facet joint, wherein more than one half of each drilled hole is contained within its respective bone.

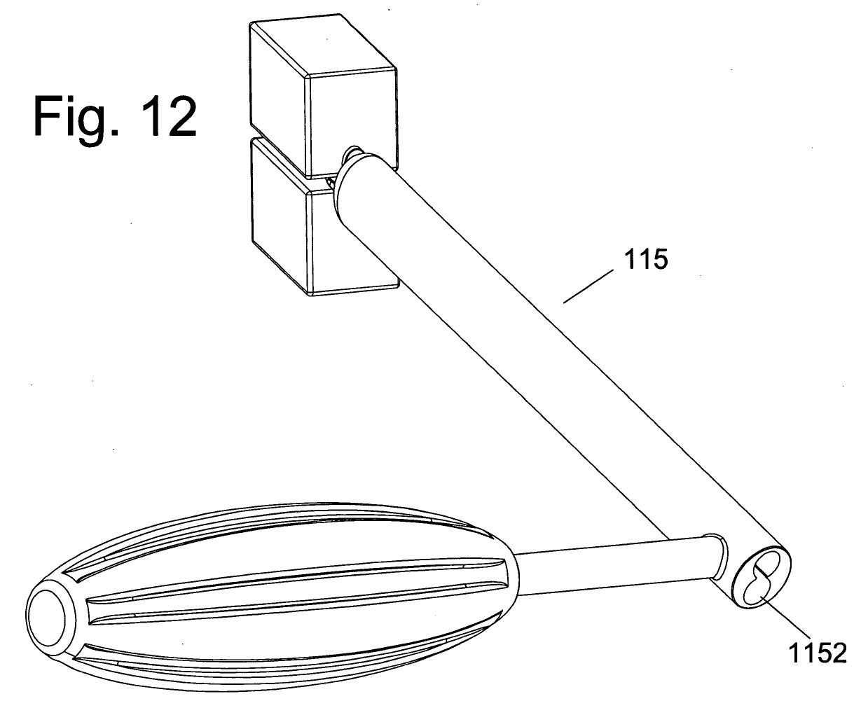

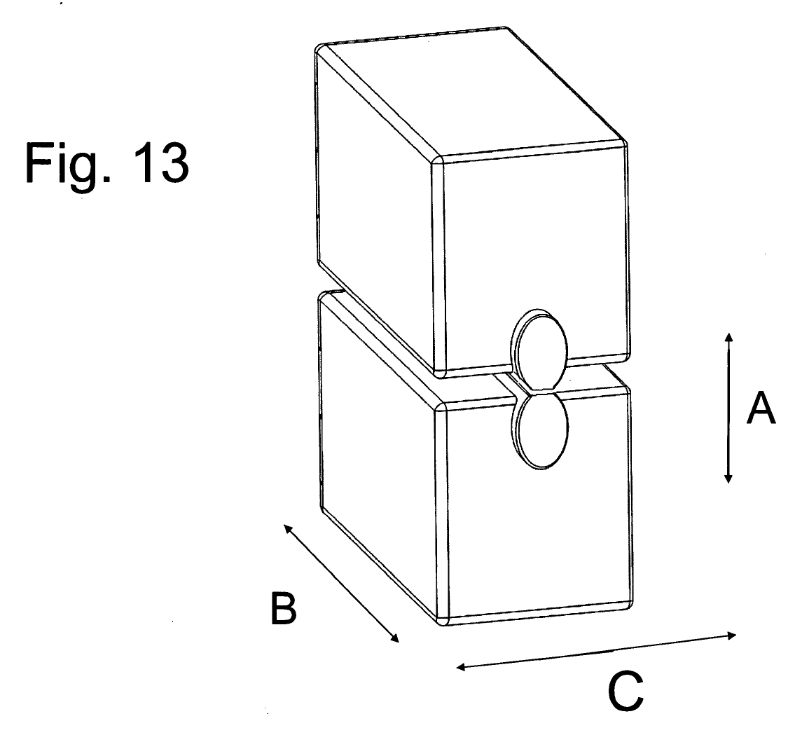

FIG. 11 shows the instrument 115 removed from the joint 105 so that the bone holes are viewable. In actual use, the instrument remains attached to facet joint 105, as shown in FIG. 12, until the procedure is completed.

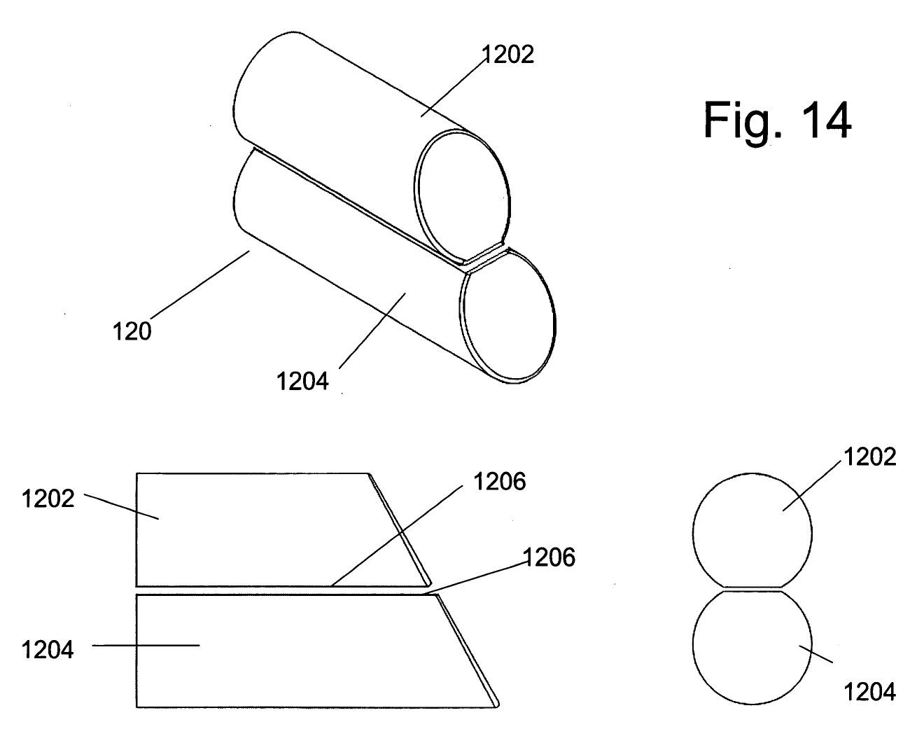

After the bone holes have been created, the bores 1152 of the instrument 115 serve as a conduit for placement of a prosthesis into facet joint 105. After delivery of the implant 120 or prosthesis, the instrument 115 is removed leaving the implanted joint. FIG. 13 shows the implant 120 with the instrument removed. Since more than one half of each drilled hole is contained within its respective bone, an implant that is press fitted within a bone hole will be effectively retained in that position without dropping into the joint space. Further, the bone holes may be cut in a substantially conical (instead of a cylindrical) configuration so that the sides of the bone holes angle inward toward the base/bottom of the hole. In addition, the sides of the prosthesis that abut the angled walls of the conical cut are preferably angled at an angle different from that of the walls so as to form a Morse taper and/or interference fit with the bone hole surfaces. The surface of the implants that contacts the bone may be threaded or ridged into order to increase the extent of fixation. The implant surface may be also coated/made with osteo-conductive (such as deminerized bone matrix, hydroxyapatite, and the like) and/or osteo-inductive (such as Transforming Growth Factor “TGF-B,” Platelet-Derived Growth Factor “PDGF,” Bone-Morphogenic Protein “BMP,” and the like) bio-active materials that promote bone formation. Further, they may be made with a porous ingrowth surface (such as titanium wire mesh, plasma-sprayed titanium, tantalum, porous CoCr, and the like), provided with a bioactive coating, made using tantalum, and/or helical rosette carbon nanotubes (or other carbon nanotube-based coating) in order to promote bone in-growth or establish a mineralized connection between the bone and the implant, and reduce the likelihood of implant loosening. Finally, the implant may be at least partially made out of bone.

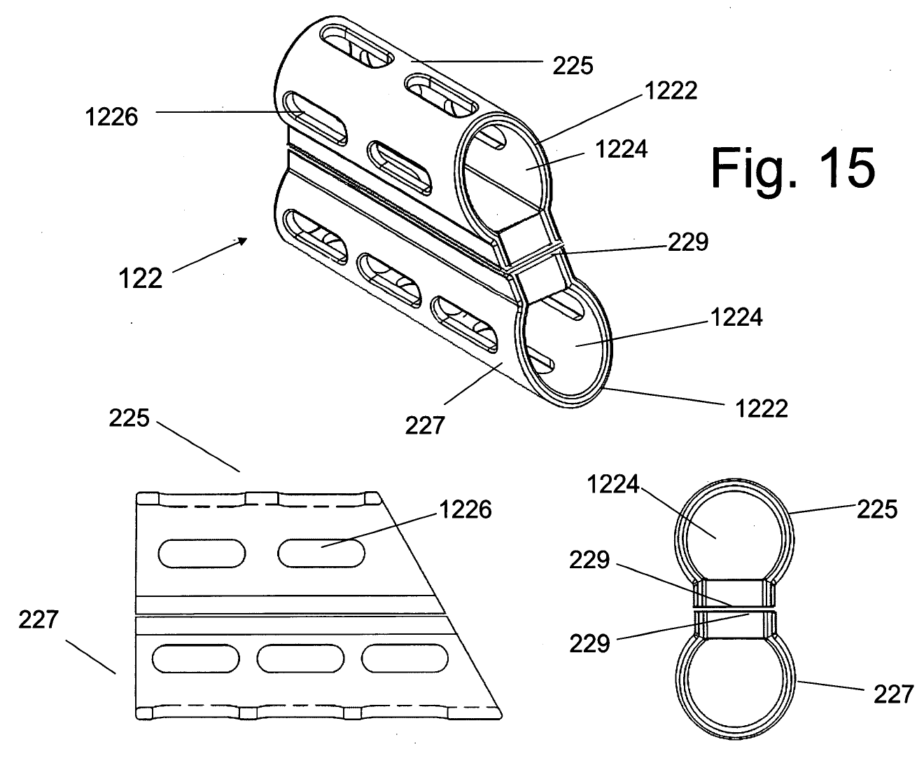

FIG. 14 shows multiple views of the implant 120. The implant 120 includes an upper segment 1202 and a lower segment 1204. In an embodiment, each of the upper and lower segments has a substantially cylindrical shape with an inclined surface at one end. The upper segment 1202 and lower segment 1204 abut one another at surfaces 1206 and permit movement of the vertebral facet joint in each of directions A, B, and C (shown in FIG. 13). The illustrated prosthesis permits motion in all three planes, including rotation. Alternatively, the prosthesis may be easily fitted with at least one motion limiting feature so that motion is limited in at least one plane. Further, a malleable member (such as a tether, spring, elastomer, and the like) may be attached to segments 1152 and/or 1154 to bias the motion towards a specific position (“neutral”) and return the joint to that position after a force acting upon the functional spinal unit (FSU) has dissipated.

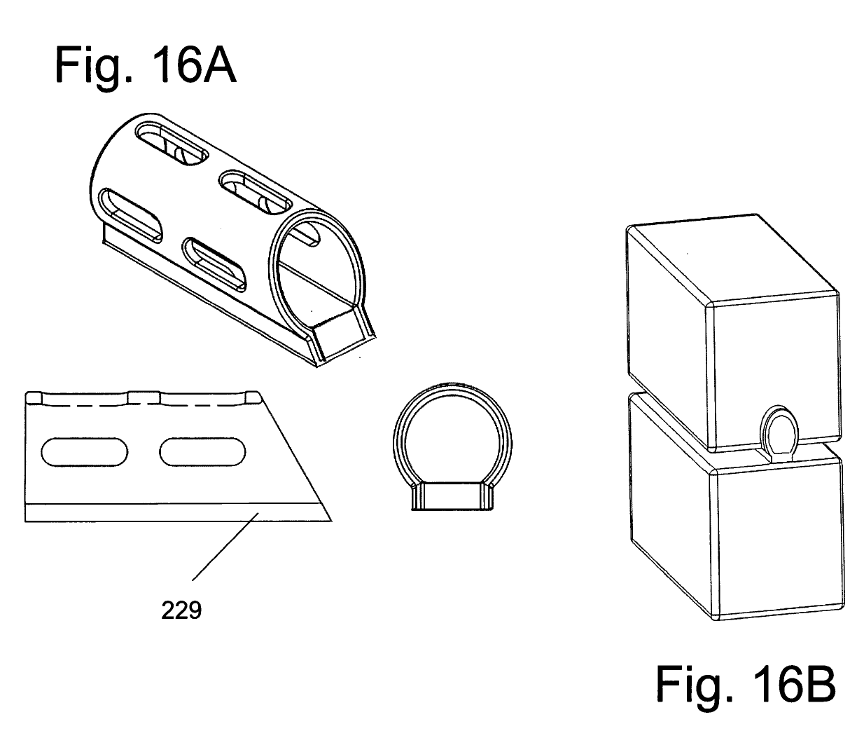

FIG. 15 shows an alternate embodiment of the implant 122. The implant 122 has an upper segment 225 and lower segment 227 that abut one another at surfaces 229 and permit movement of the vertebral facet joint in all three planes, including rotation. As in the previous embodiment, each segment has a substantially cylindrical shape although the segments in this embodiment are hollow. Each of the upper and lower segments has a solid outer wall 1222 with a hollow central cavity 1224 that may be filled, at least partially, with bone graft or bone graft substitute (collectively referred to as bone graft material). On implantation, holes 1226 on the outer wall 1222 of each implant segment will permit the material contained within cavity 1224 to fuse with the bone outside of each implant segment. In this way, each prosthesis segment is rigidly affixed to the vertebral bone in which it is embedded (for example, segment 225 is fused to upper vertebra and segment 227 is fused to the lower vertebra), while motion is still maintained across the facet joint at least partially through abutment surfaces 229.

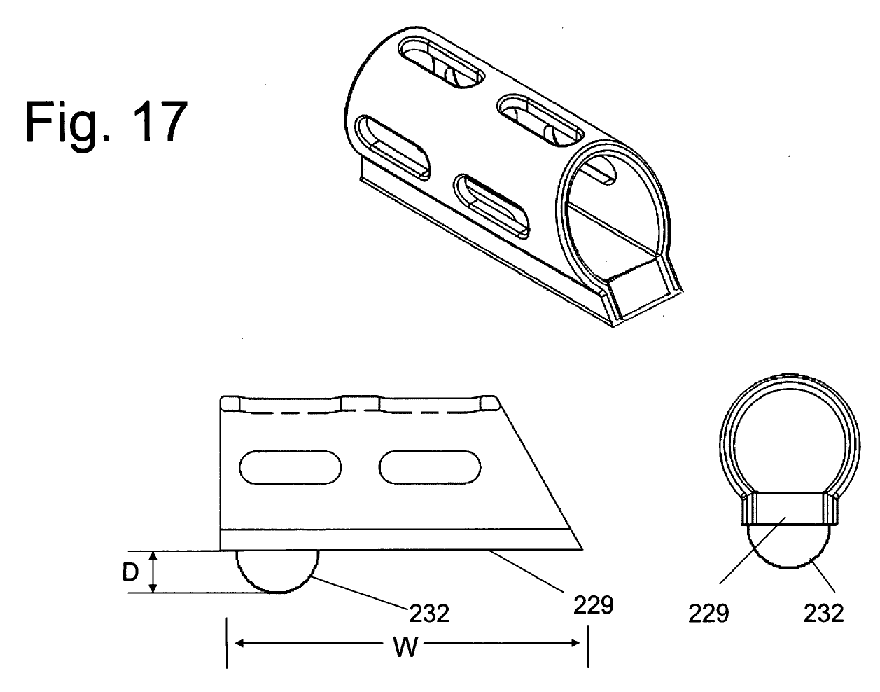

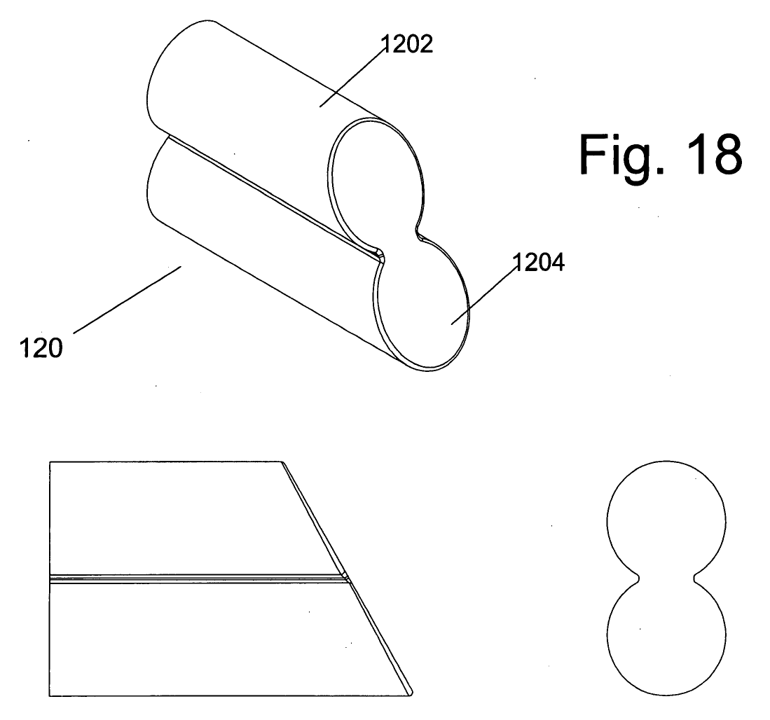

In an alternative embodiment, the instrument 115 may be adapted with a single bore 1152 and used to place a single bore hole into either side of joint 105. FIG. 16A illustrates a device embodiment wherein an implant 225 is placed into one vertebral body adjacent to the facet joint. Unlike the previous embodiments, the implant 225 has a single segment rather than upper and lower segments. The implant 225 includes an abutment member or surface 229 that abuts a vertebral surface adjacent to the vertebra into which the implant 225 is implanted and fused. In this embodiment, the implant has an abutment surface 229 that directly abuts the joint surface of the adjacent vertebra. FIG. 16B shows the implant of FIG. 16A in an implanted state. (While shown attached (and fused) to the upper vertebra and abutting the lower vertebra, it is understood that the implant may be alternatively adapted to attached (and fused) onto the lower vertebra and abut the upper vertebra.)

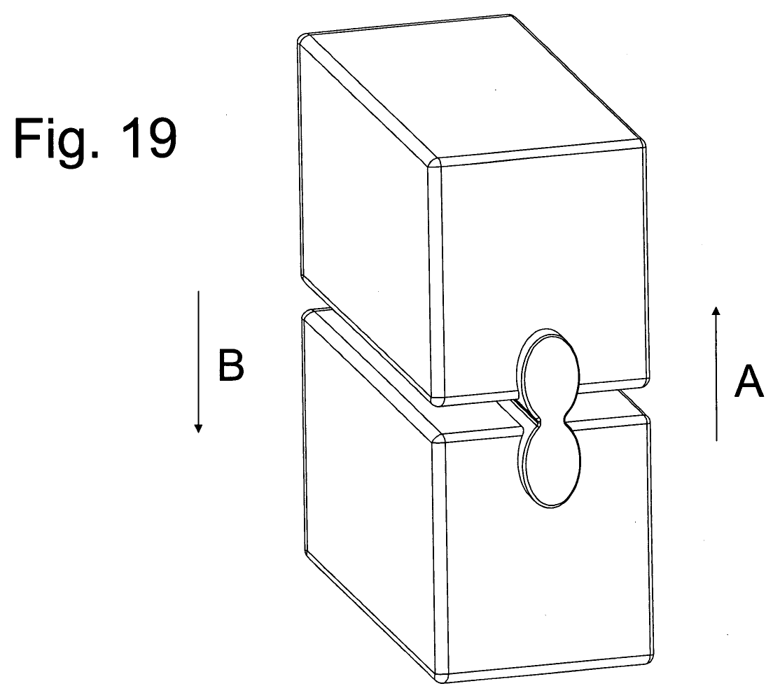

FIG. 17 shows an additional embodiment of the implant that includes a protrusion surface 232 shaped as a segment of a curvilinear surface. The protrusion is added onto abutment surface 229 of the embodiment of FIG. 16B. The surface 232 provides an abutment surface with the bone surface of the adjacent vertebra. The surface 232 may be placed towards the anterior aspect of the implanted prosthesis (as shown in FIG. 17) so that the abutment surface is closer to the center of vertebral rotation. While not depicted, the embodiment of FIG. 17 may be further adapted by one of ordinary skill in the art to include a movable surface 232, wherein the position of the surface may be varied along the direction “W” or direction “D”.

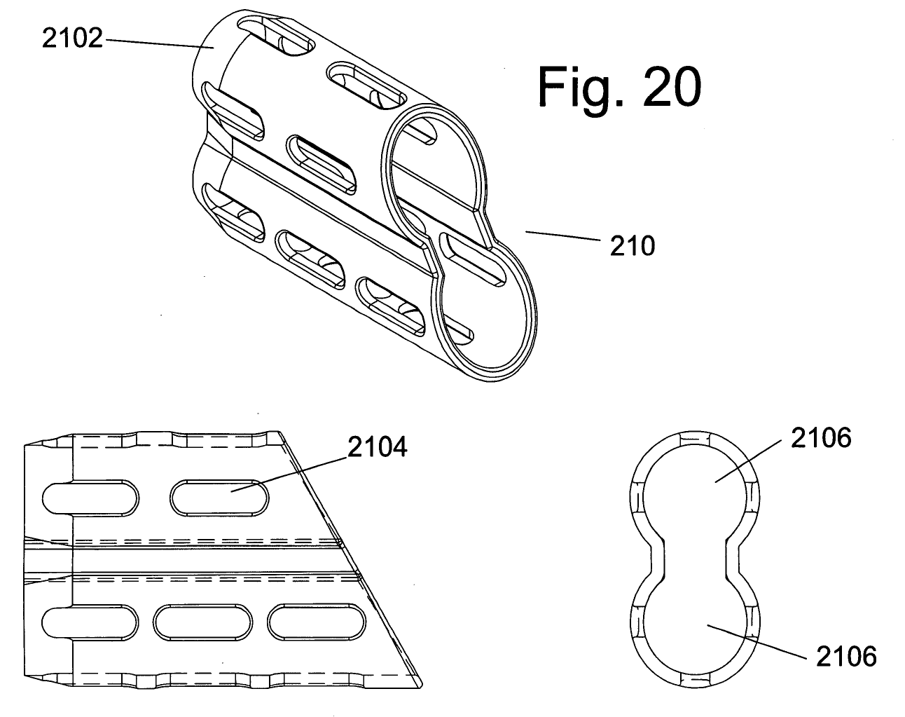

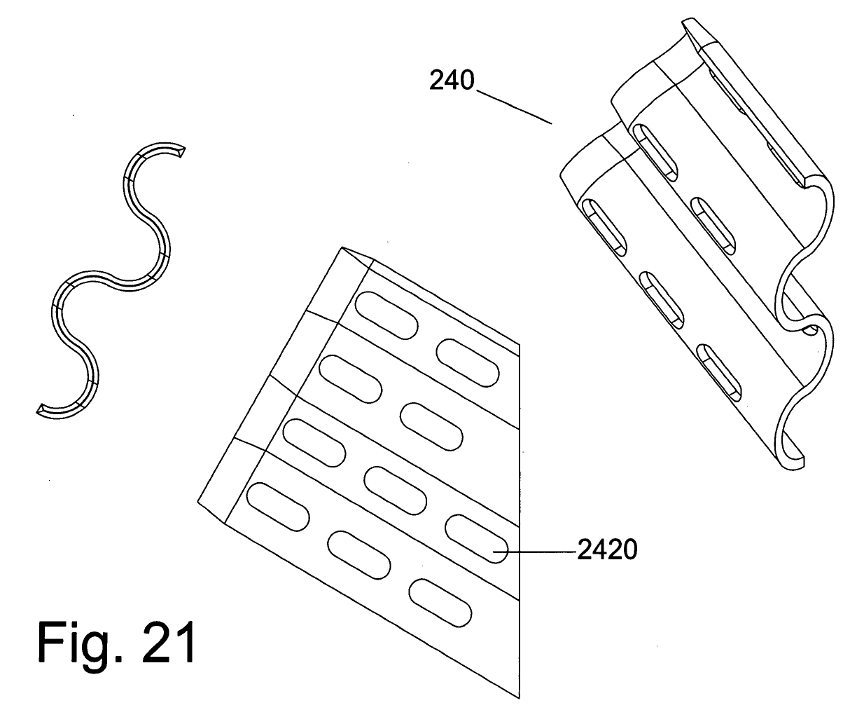

FIG. 18 shows an alternative embodiment of an implant. In this embodiment, upper segment 1202 and lower segment 1204 are interconnected and, with implantation, the device can immobilize facet joint 105. FIG. 19 shows the implant of FIG. 18 in an implanted state. Since more than one half of each drilled hole is contained within the bone, the prosthesis provides resistance to movement in both flexion (direction A) and extension (direction B) of the spine. While it may be made of any biologically implantable material, the prosthesis is preferably comprised, at least partially, of bone and/or a bone graft substitute so that bone healing and fusion may occur across the joint and rigidly affix the upper and lower bones to one another.

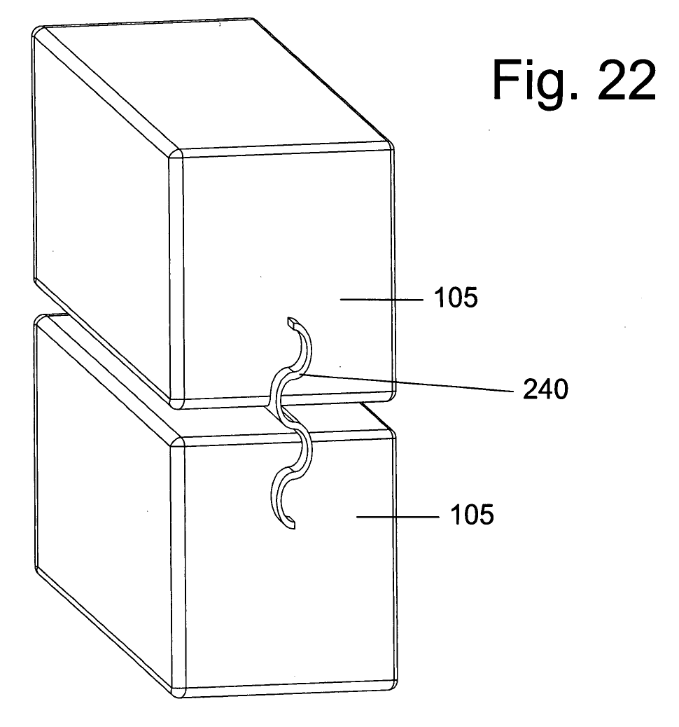

FIG. 20 shows an additional embodiment of the implant. The implant 122 of FIG. 20 has a solid outer wall 1222 with hollow central cavity 1224 that may be filled, at least partially, with bone graft or bone graft substitute. On implantation, holes 1226 on the outer wall 1222 of implant 122 permit the material contained within cavity 1224 to fuse with the bone outside of implant 122. In this way, a bony fusion occurs across the prosthesis and rigidly affixes the upper and lower bones to one another.

In an alternative use of the prosthesis, the device is placed through bores 1152 of instrument 115 and driven into the intact bone without pre-drilling a bore hole into each bone. The sharp leading edge 2102 of prosthesis 210 functions like a chisel forcing a segment of bone into each of central cavities 2106. The bone segments contained within cavity 2106 will fuse with the surrounding vertebral bone across the bore holes 2104 contained within the prosthesis wall. While the bone segments contained in cavity 2106 may also fuse with each other, it is possible that they would not do so because the cartilaginous material of the joint space between them had not been removed. In the current art, fusion of two bones requires that a bony bridge be directly formed from one bone to the other. However, in this embodiment, the upper segment contained in 2106 is fused with the upper vertebral bone and the lower segment contained in 2106 is fused with the lower vertebral bone but no bony bridge is directly formed between the two segments. The vertebral bodies are immobilized relative to one another by the rigid prosthesis and not as a result of a direct bony fusion between them. That is, the prosthesis is immobilized relative to each of the two bones because of the formation of a bony bridge across the prosthesis wall and the vertebral bones are immobilized relative to one another because of the action of the rigid prosthesis wall.

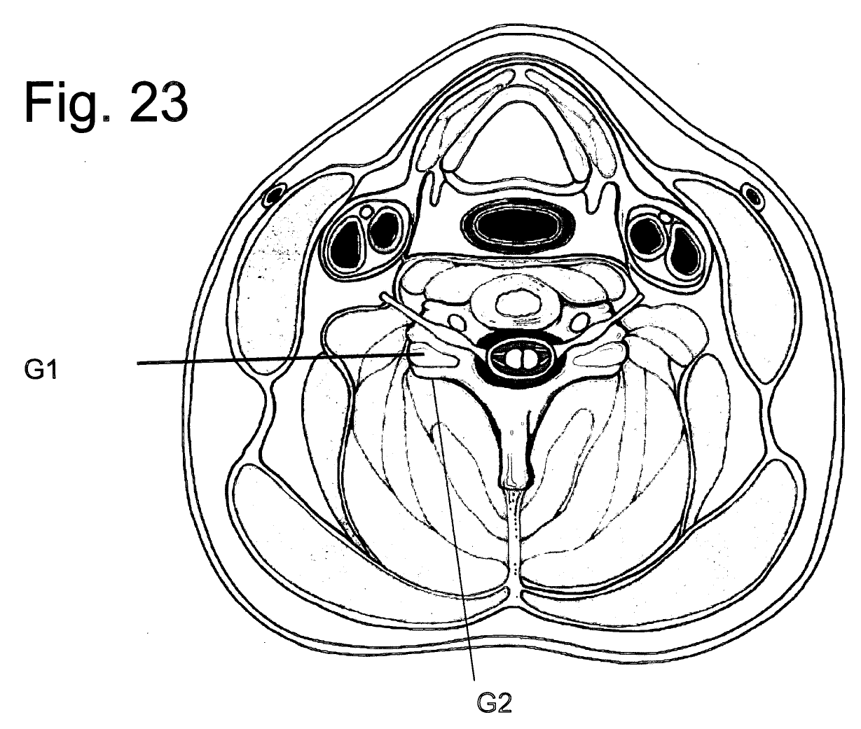

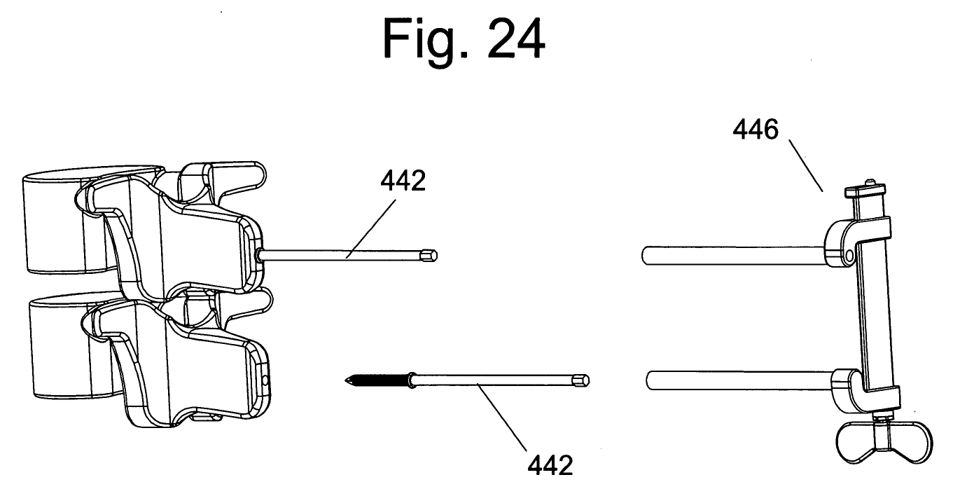

FIG. 21 shows various views of an alternate embodiment of an implant 240. The implant 240 has an undulating or corrugated shape and also has a tapered width that increases moving along one dimension of the implant. FIG. 22 shows how the implant of FIG. 21 is implanted between the facet joints 105. The implant 240 is driven across the joint and into the underlying bone. As shown in FIG. 21, the implant 240 may include multiple holes 2420 that extend through the implant 240. The holes 2420 permit bone growth across the prosthesis and increase the fixation power of the prosthesis.

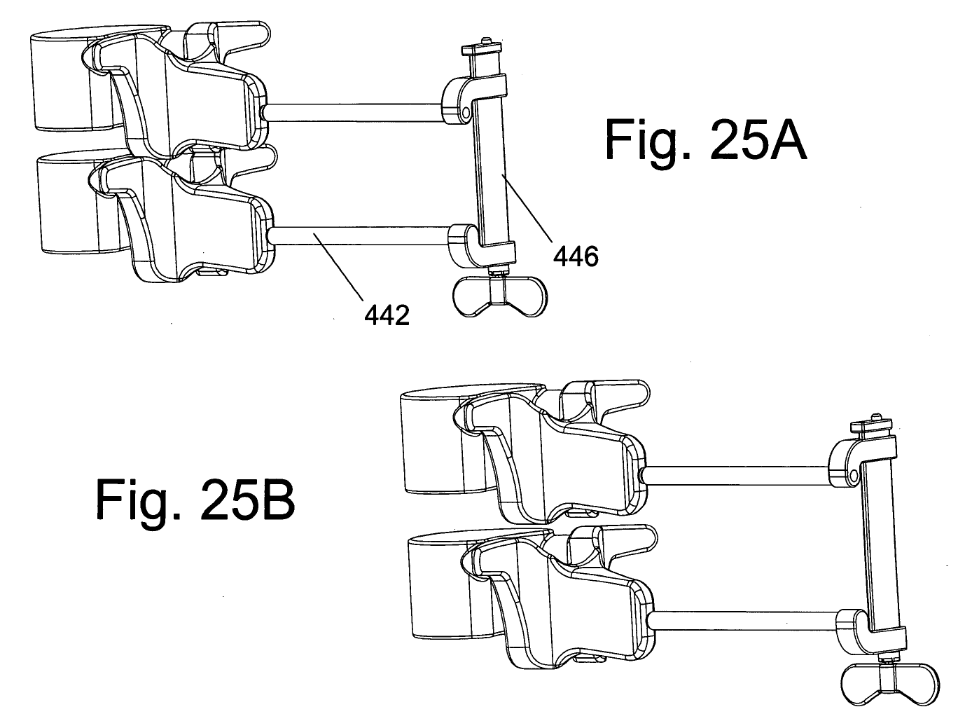

FIG. 23 shows a schematic representation of a cross section of the neck. Those skilled in the art will appreciate that an actual cross section of the neck may include anatomical details that are not shown in FIG. 23. It is understood that the aforementioned embodiments and the disclosed methods of implantation may be used through a substantially posterior approach (represented by G2 in FIG. 23), a substantially lateral approach (represented by G1 in FIG. 23), or any approach corridor in between.

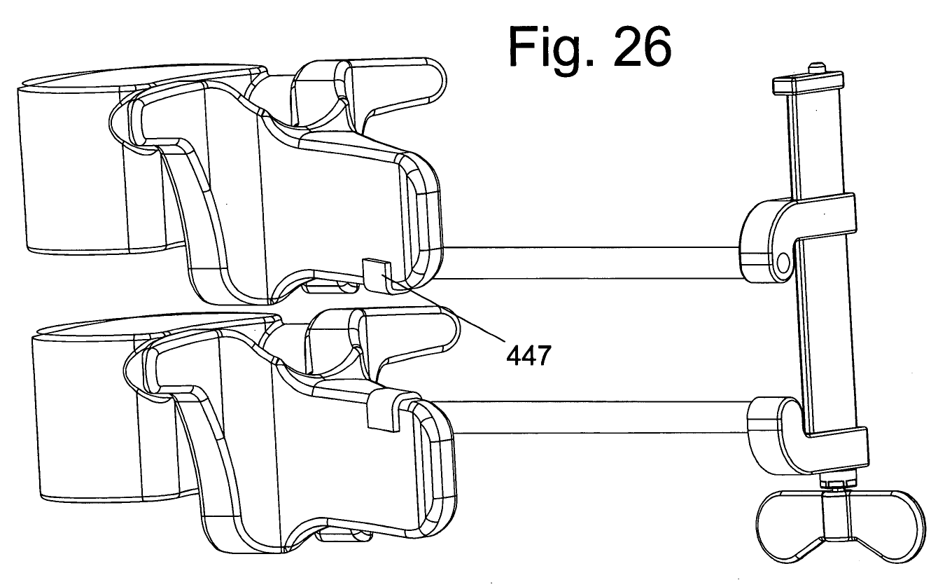

Studies of diseased facet joints have shown that the superior articulating process of the lower vertebra is usually the more diseased segment of the facet joint. Because of its proximity to the nerve roots, osteophytes and other degenerative outgrowths of the superior articulating process of the lower vertebra commonly produce nerve root compression. Effective decompression of the nerves can be accomplished by removal of at least a portion of the joint, and preferably, the resected segment would include at least a portion of the superior articulating process of the lower vertebra. However, because the superior articulating process of the lower vertebra is located anterior to the inferior articulating process of the upper vertebra, it is not currently possible to remove the former without concurrently injuring the latter. FIGS. 24-26 illustrate a method for the selective removal of at least a portion of the superior articulating process of the lower vertebra and its subsequent replacement with a orthopedic implant. In this way, the facet joint is partially replaced.

The procedure is started with the distraction of the vertebral bones. With reference to FIG. 24, at least one distraction screw and/or pin 442 is anchored into each vertebral bone. A distal end of the pin 442 is anchored into the bone such that the pin 442 extends from the bone. A pin 442 is anchored into each of adjacent bones. The pins 442 are adapted to couple to a distraction platform 446 that can be used to apply a distraction force to the pins 442 and attached bones. In a next step of the procedure, a distraction force is applied to the pins 442 using the platform 446 so that the bones (vertebrae) are moved apart. The pins 442 may optionally be placed into the spinous process segment of the vertebral bones as shown in FIG. 24. Alternately, the pins 442 may be placed in different locations in the bones, such as the pedicle portions of the vertebras. For clarity of illustration, the vertebral bones are represented schematically and those skilled in the art will appreciate that actual vertebral bones may include anatomical details not shown in FIG. 24.

Next, the joint capsule on each facet joint is incised in order to facilitate vertebral distraction. Alternatively, the joint capsule is left intact and is not incised prior to distraction. As shown in FIGS. 25A and 25B, the distraction platform 446 is used in conjunction with the pins 442 to distract the vertebral bones and open the facet joint on each side of the vertebral midline. The distracted joint permits unhindered access to the superior articulating process of the lower vertebra and allows removal of the diseased segments without injury to the inferior articulating process of the upper vertebra.

FIG. 26 illustrates additional method of attaching the distractor platform 446 to the bones. In this embodiment, retention arms or hooks 447 may be alternatively used to distract the bones without the use of distraction screws. Further, vertebral distraction may be alternatively accomplished through attachment and/or abutment of the instruments producing the distraction force to any applicable point on the vertebral bone, such as, for example, the laminas.

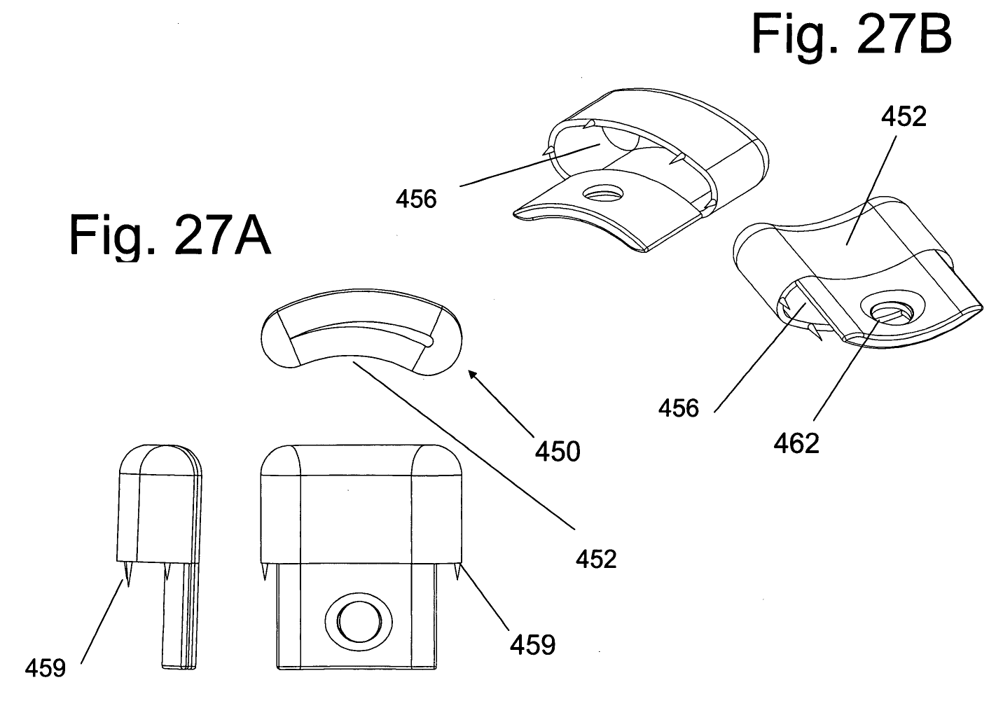

After removal of the diseased segments of the articulating processes and decompression of the underlying nerves, a replacement prosthesis may be attached onto the lower vertebra and used to reestablish the articulation between the vertebra. FIGS. 27A and 27B show various views of an exemplary replacement prosthesis 450. The prosthesis 450 is a wing-shaped member having an outer articulation surface 452 adapted to articulate with the inferior articulating process of the upper vertebra. A cavity 456 is preferably located within the prosthesis 450, wherein the cavity 456 is adapted to be filled with bone graft or bone graft substitute so that a bony fusion may be formed with the cut surface of the superior articulating process of the inferior vertebra. The prosthesis 450 may contain additional features (such as, for example, spike protrusions 459) that enhance device attachment onto the vertebral body to which the device is rigidly attached. The prosthesis 450 may include one or more bores 462 adapted to accept a bone screw 464, wherein the bone screw anchors onto the inferior vertebra. An interference fit may be formed between the head of the screw and the bore 462 so that, with full seating of screw, the screw head is immobile relative to the prosthesis 450.

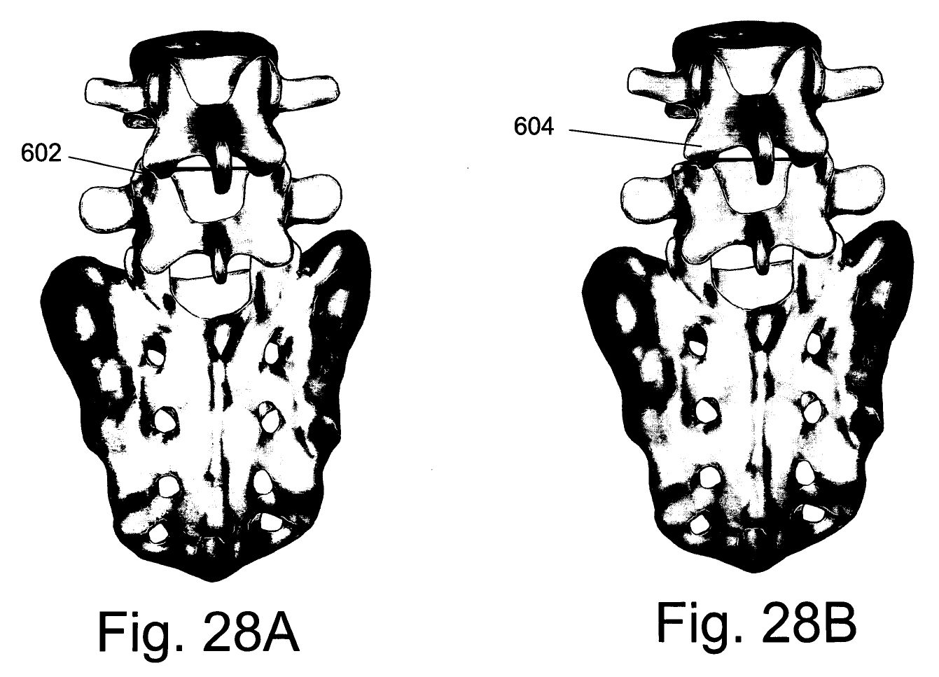

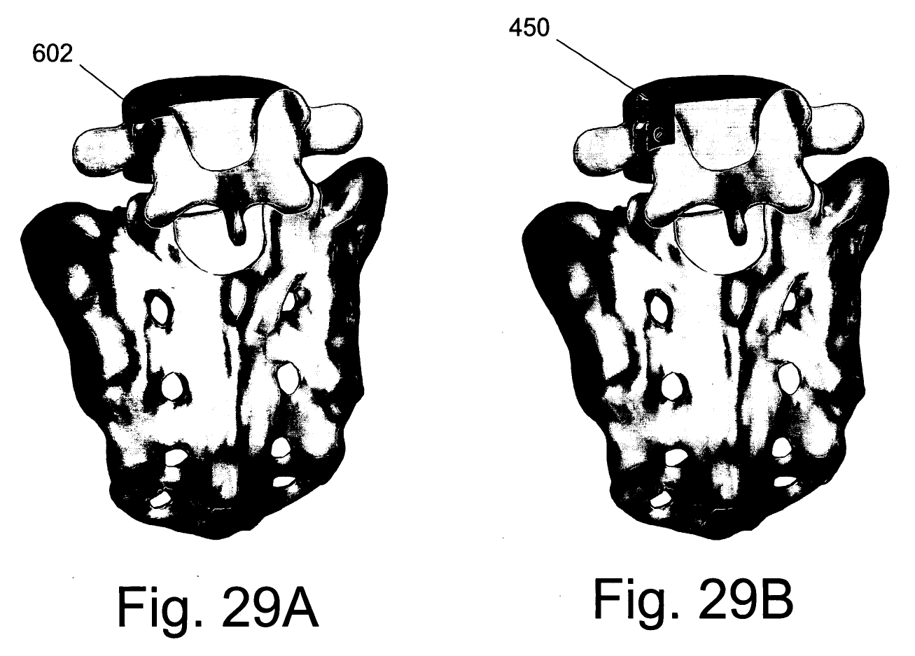

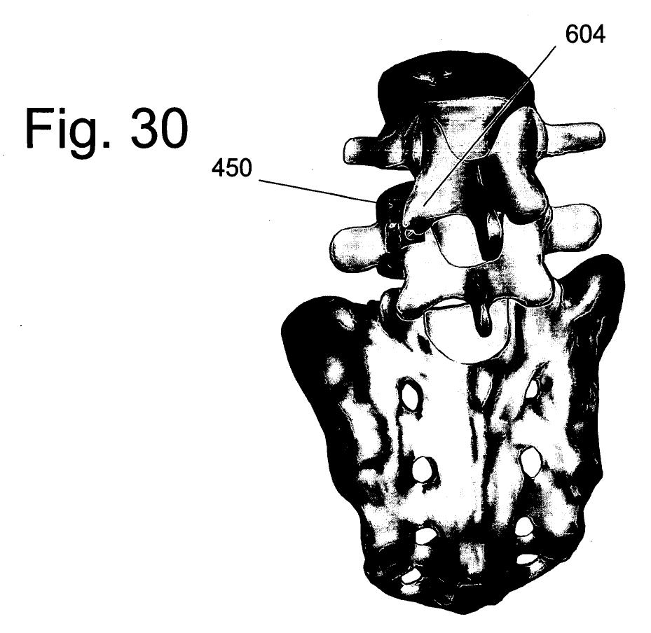

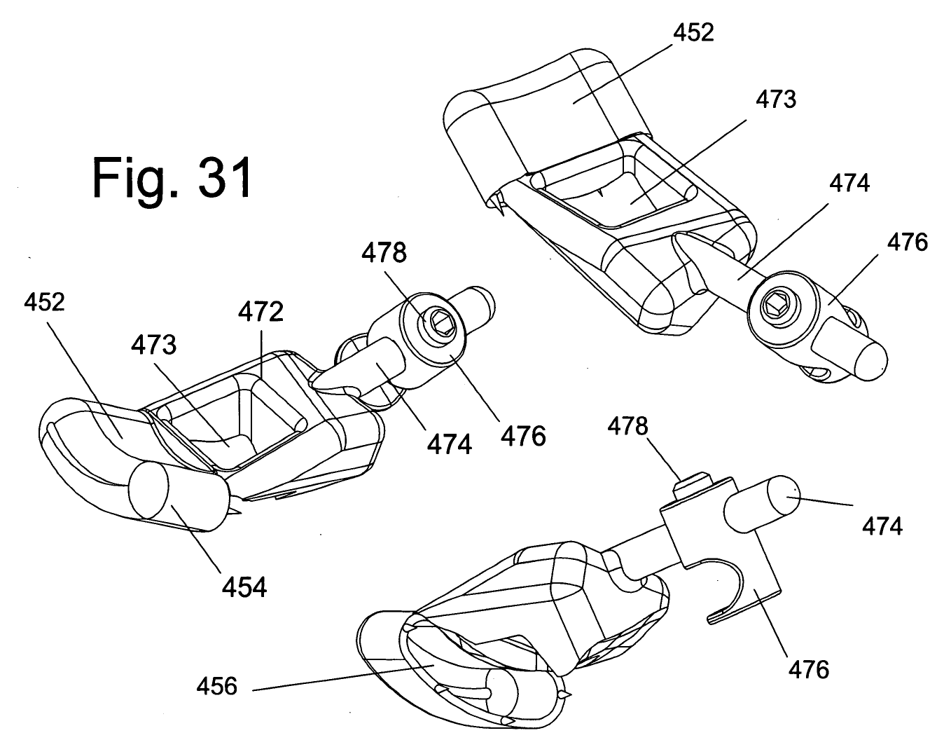

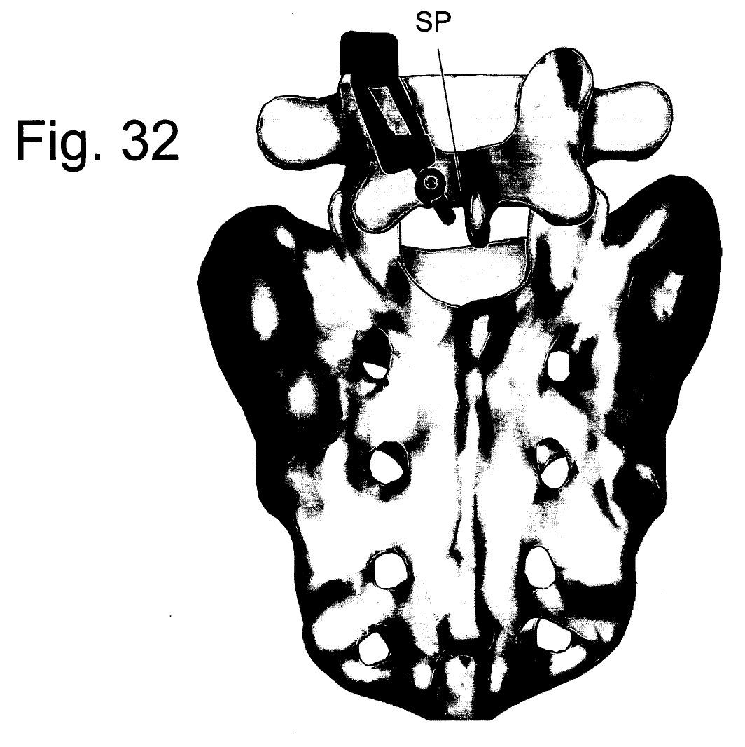

FIG. 28A illustrates a spinal segment prior to distraction while FIG. 28B shows the distracted spinal segment after removal of the diseased portion of the superior articulating process 602 of the lower vertebra. Note that the overlying inferior articulating process 604 of the upper vertebra has been safely distracted and protected from injury during removal of the diseased portion of the superior articulating process 602. FIG. 29A shows the distracted spinal segment with the superior vertebra removed in order to better demonstrate the cut surface of the superior articulating process 602. FIG. 29B shows the prosthesis 450 in an implanted state. The distraction is removed after placement of the prosthesis 450 and the articulation/joint is reestablished. FIG. 30 shows the spine after placement of the prosthesis 450 and removal of the distraction.

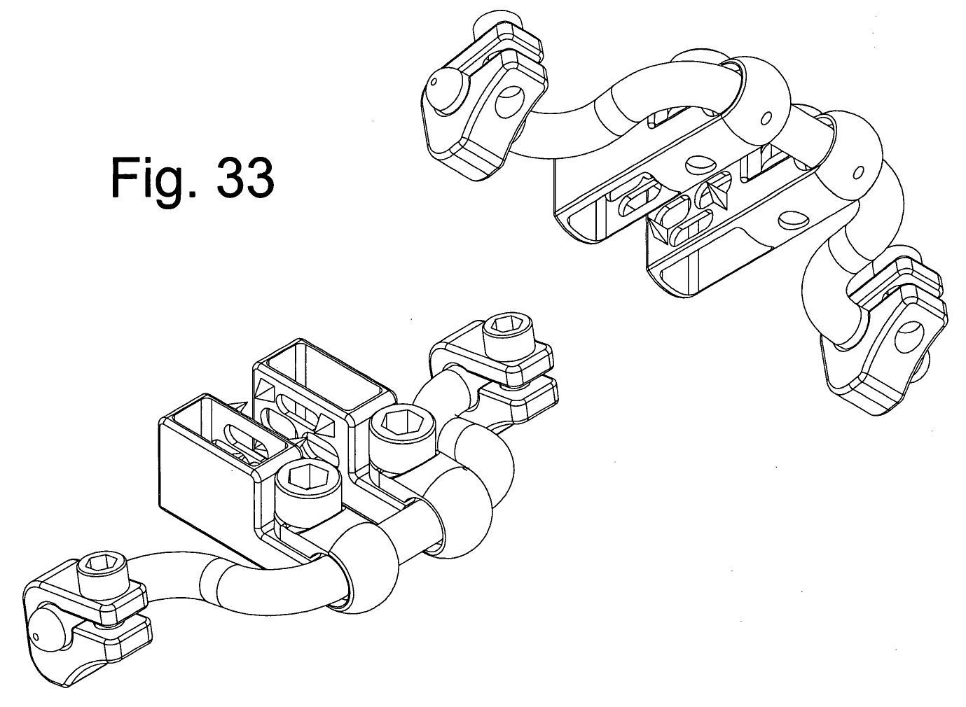

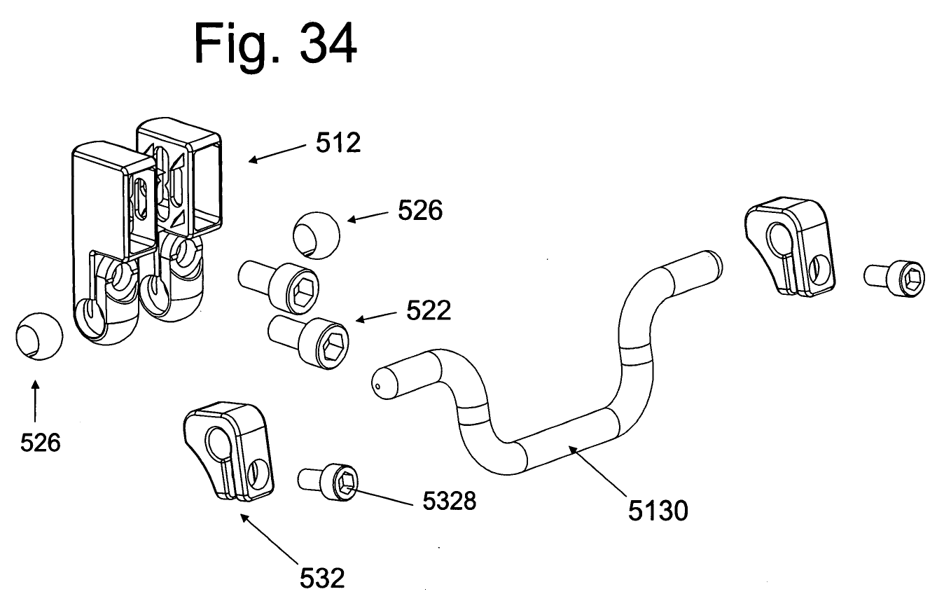

FIG. 31 shows an alternative embodiment of an implant. The implant includes a body member 454 that is similar to the device shown in FIGS. 27A and 27B. In this regard, the body member 454 includes an internal cavity 456 and an outer articulation surface. In addition, the prosthesis includes a lamina member 472 that is sized and shaped to abut a lamina. The lamina member 472 has a cavity 473 adapted to be filled with bone graft or bone graft substitute so that a bony fusion may be formed between the device and the underlying bone. A rod member 474 is attached to one end of the lamina member 472 opposite the body member 454. The rod member 474 is attached to a lamina hook 476 adapted to anchor the device onto the lamina of the inferior vertebra. A set screw 478 can be used to rigidly affix the hook 476 onto the rod member 474. FIG. 32 shows the prosthesis of FIG. 31 attached to bone. Note the lamina member 472 and cavity 473 may be enlarged so as to extend over a larger segment of the lamina surface or even the lateral aspect of the spinous process (surface SP).

The preceding disclosure has illustrates replacement of at least a portion of the superior articulating process of the inferior vertebra and maintain motion between an adjacent superior and inferior vertebral bones. A first segment of the device is rigidly attached to at least a segment of a the inferior vertebra and a second segment of the device forms an abutment surface with at least a segment of an inferior articulating process of the superior vertebra (or a prosthesis adapted to replace it). Further, the first device segment contains a cavity that is adapted to house a bone forming material and to form a bony fusion with a bony segment of the inferior vertebra. The second device segment is adapted to abut but not rigidly affix onto or fuse with at least a portion the inferior articulating process of the superior vertebra or with a prosthesis adapted to replace at least a portion of that segment of the superior vertebra.

A comparable device can be configured to replace at least a segment of the inferior articulating process of the superior vertebral bone. While not specifically illustrated by drawings, this device follows the same design principle the preceding embodiment. In this implant, a first segment of the device is rigidly attached to at least a segment of a the superior vertebra and a second segment of the device forms an abutment surface (preferably, the abutment surface is a portion of a sphere) with at least a segment of a superior articulating process of the inferior vertebra (or a prosthesis adapted to replace it). Further, the first device segment contains a cavity that is adapted to house a bone forming material and to form a bony fusion with a bony segment of the superior vertebra. The second device segment is adapted to abut but not rigidly affix onto or fuse with at least a portion the superior articulating process of the inferior vertebra or with a prosthesis adapted to replace at least a portion of that segment of the inferior vertebra.

The implanted devices serve to limit translation of the superior vertebra relative to the inferior vertebra in the transverse plane and may be also used to reduce the extent of anterior spondylolisthesis between the two adjacent vertebrae. Further, the devices may be positioned so that the functional spinal unit (FSU) is placed into slight anterior flexion. This vertebral re-alignment would limit extension and enlarge the cross-sectional area of the spinal canal.

In another embodiment, a device is adapted to at least partially replace a portion of a lamina and both of the ipsilateral inferior and superior articulating processes of the middle vertebra of an assembly of three consecutive vertebral bones. While not specifically illustrated by drawings, this device follows the same design principle the preceding embodiment. A first segment of the device is rigidly attached to at least a portion of the residual ipsilateral pedicel of the middle vertebra, while a second segment of the device forms an abutment surface with at least a segment of a superior articulating process of the inferior vertebra (or a prosthesis adapted to replace it) and a third segment of the device forms an abutment surface with at least a segment of an inferior articulating process of the superior vertebra (or a prosthesis adapted to replace it). Further, the first device segment contains a cavity that is adapted to house a bone forming material and to form a bony fusion with at least a portion of the residual ipsilateral pedicel of the middle vertebra. The second device segment is adapted to abut but not rigidly affix onto or fuse with at least a segment of a superior articulating process of the inferior vertebra while the third device segment is adapted to abut but not rigidly affix onto or fuse with at least a segment of an inferior articulating process of the superior vertebra. Alternatively, either second or third segments may be adapted to affix onto and fuse with at least a segment of the complimentary articulating process of the adjacent vertebra. In this way, the construct of the three consecutive vertebrae would include a first pair of adjacent vertebral bones that are fused and immobile relative to one another and a second pair of adjacent vertebral bones that are mobile relative to one another.

The embodiments of FIGS. 27-32 disclose selective removal of at least a portion of the superior articulating process of the inferior vertebra and subsequent replacement of the articulation with a prosthesis. The tissue resection is preferably, but not necessarily, performed after distraction of the vertebral bones and disengagement of the facet joints. This allows the selective removal of the diseased segment of the superior articulating process of the inferior vertebra without violation of the inferior articulating process of the superior vertebra.

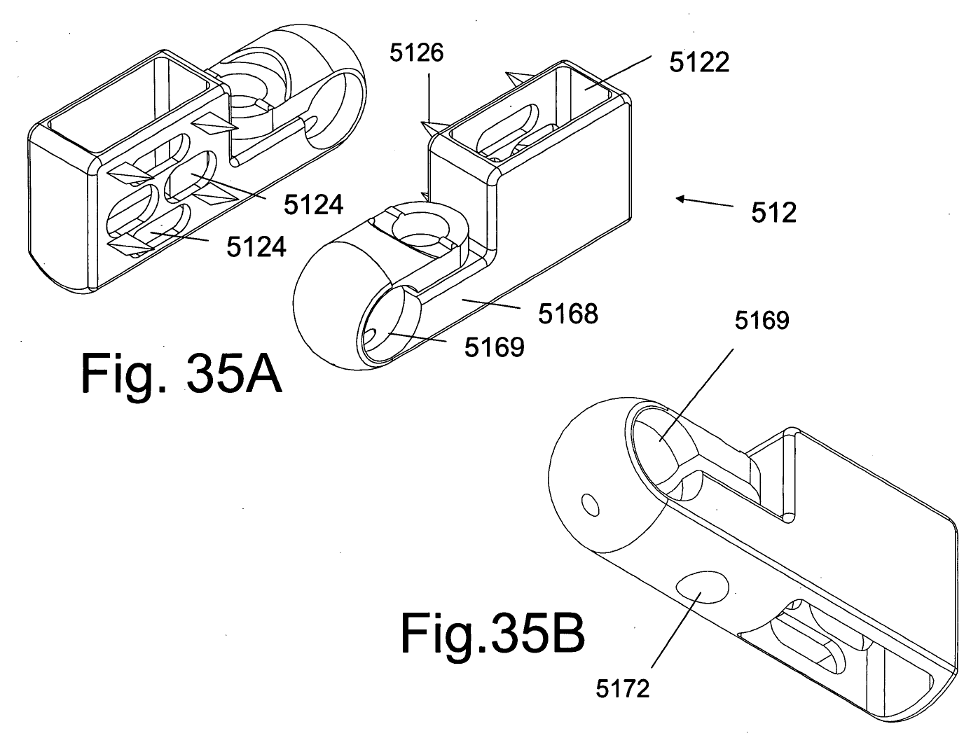

An additional embodiment is now disclosed, wherein the device is adapted to form an additional articulation between the superior and inferior vertebra without resection and/or replacement of segments of the anatomical facet joints. The new articulation is produced by the rigid attachment of a device onto the superior vertebra, wherein the device contains a cavity adapted to accept bone graft or bone graft substitute (collectively referred to as bone graft material) that will form a direct bony fusion with a surface of the superior vertebra. The device further contains a surface adapted to abut and articulate with a segment of the inferior vertebral bone, wherein, preferably, the device is not directly anchored to the inferior vertebra and the abutment surface does not directly articulate with a segment of the articulation surface of the facet joint. An exemplary illustration is shown in FIG. 33.

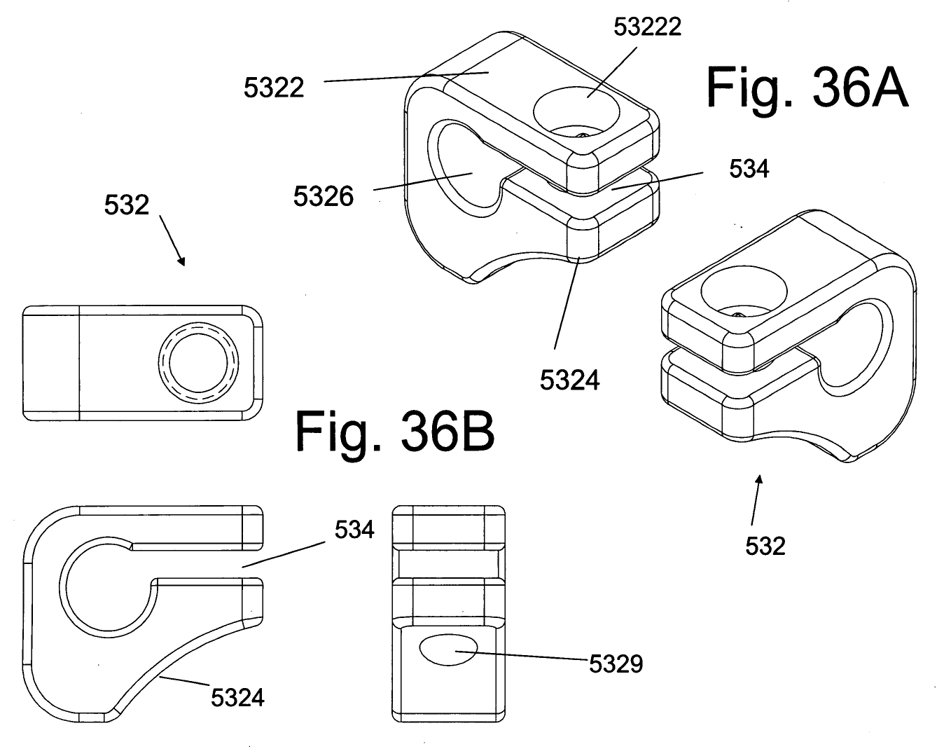

The device contains at least one cavity adapted to contain a bone graft material that fuses with the spinous process and/or lamina of superior vertebral bone (FIG. 33). The device further contains an abutment surface that is adapted to abut the superior and/or posterior aspects of the superior articulation process of the lower vertebral bone, wherein, preferably, the joint capsule of the facet joint remains substantially intact. FIG. 34 shows an exploded view of the device of FIG. 33.

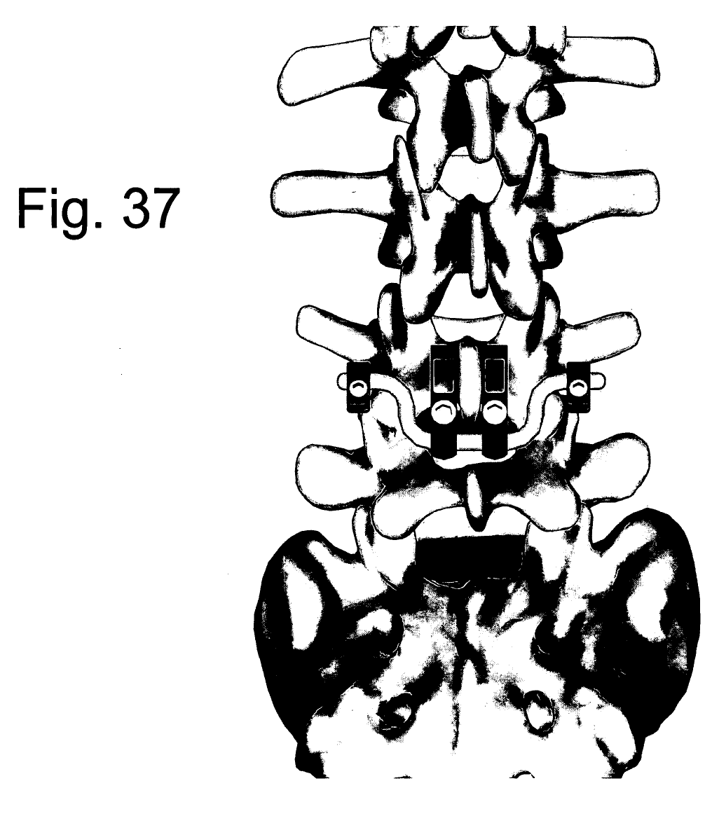

FIGS. 35A and 35B illustrate multiple perspective views of the member 512 of the device of FIG. 33. The member 512 is substantially L-shaped and includes a main section with an internal compartment 5122 that is adapted to receive and house a bone graft or bone graft substitute. The main section includes multiple bores 5124 of variable size through the medial wall and/or bottom wall that borders the compartment 5122. The bores 5124 permit communication between the bone graft material within compartment 5122 and the adjacent spinal bone, so that a bony fusion could be established between the bone graft within compartment 5122 and the adjacent spine. The member 512 also includes multiple spiked protrusions 5126 that permit device fixation to the adjacent bone. The member 512 further includes a segment 5168 that is split along a portion of itself. The segment 5168 defines a central bore 5169 that can be adjusted in size by virtue of one portion of the split segment 5168 moving relative to another portion along the split. A locking screw can reside within a threaded bore 5172 of member 512.

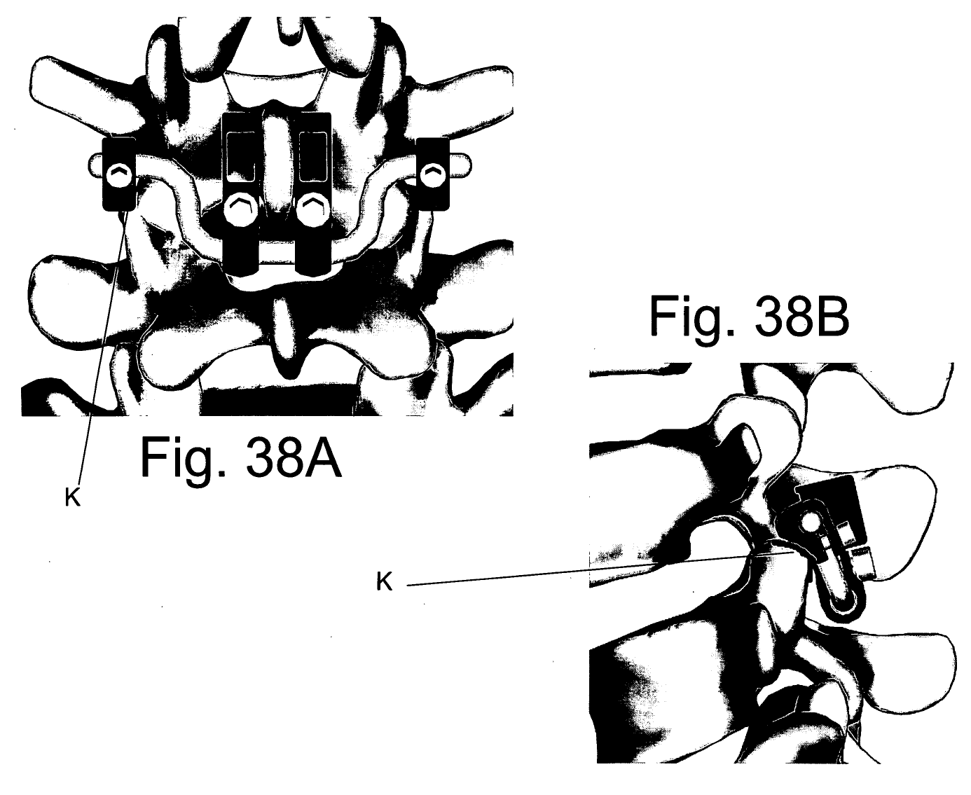

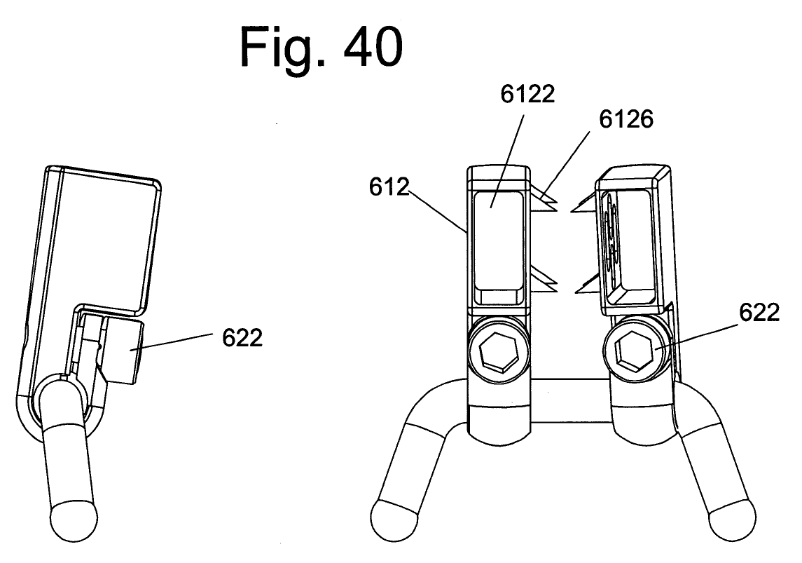

FIGS. 36A and 36B show multiple views of an abutment member 532 of the device of FIG. 33. The abutment member includes a top surface 5322 having a non-threaded bore hole 53222 there through. An abutment surface 5324 is adapted to abut the superior surface and/or posterior surfaces K of a superior facet joint of the lower vertebra. Note that the abutment surface K (FIG. 38) of the superior articulation process of the inferior vertebral is outside the facet joint capsule and is not a segment of the facet joint. A bore 5326 is sized and shaped to accept bar 5130. The member 532 includes a split 534 that separates the segment bearing top surface 5322 and the segment bearing abutment surface 5324. A threaded locking screw 5328 interacts with corresponding threaded bore hole 5329. Advancement of threaded screw 5328 into threaded bore 5329 produces closure of split 534 and reduction of the diameter of bore 5326 bearing bar 5130. In this way, abutment member 532 is rigidly locked to bar 5130.

When the device of FIG. 33 is in the assembled state, a split locking sphere 526 resides within central bore 5169 of segment 5168 (FIGS. 35A and 35B). A bar 5130 resides within the central bore of the split locking sphere 526. Rotation and advancement of a locking screw 522 within threaded bore 5172 produces closure of split segment 5168 and reduction of the diameter of central bore 5169. The split locking sphere 526 is compressed and the bar 5130 is immobilized relative to the member 512. In this way the device is rigidly locked.

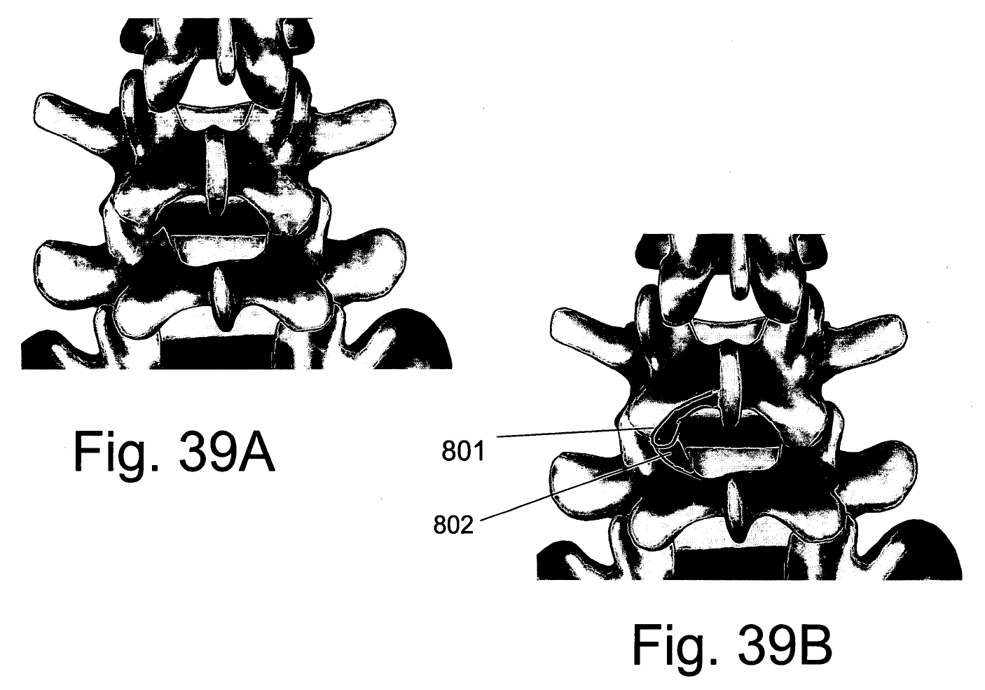

In use, the bone surface of the lateral aspect of the spinous process and/or posterior surface of the lamina are denuded of soft tissue and decorticated in preparation for bone fusion. The device is applied to the spine, wherein the bar 5130 is rotated into position so that each abutment surface 5324 of member 532 is brought into contact with surface K of its respective superior articulating process of the lower vertebra. This necessarily places the left end of bar 5130 between the left superior and inferior articulating processes of the upper vertebra and places the right end of bar 5130 between the right superior and inferior articulating processes of the upper vertebra (see FIG. 37).

Each member 512 is then forced medially by a locking tool, such as, for example, a pair of pliers so as drive spiked protrusions 5126 into the lateral aspect of the spinous process of the superior vertebra. Once positioned, each locking screw 522 is actuated so as to immobilize each member 512 relative to its bar 5130. Each locking screw 5328 is then actuated to lock abutment member 532 onto bar 5130. Bone graft material is packed into each compartment 5122, so that the bone graft material forcibly contacts the lateral wall of the spinous process and/or the posterior wall of the lamina of the superior vertebra.

In this way, the device embodiment of FIG. 33 forms an additional articulation between the superior and inferior vertebra without resection and/or replacement of segments of the anatomical facet joints. Since the implanted is rigidly attached to the superior vertebral bone and it abuts the posterior aspect of the superior articulating processes of the inferior vertebra (along surface K), the implant will resist any anterior translation of the superior vertebra relative to the inferior vertebra in the transverse (horizontal) plane. Further, the implant can be used to forcibly reduce the extent of anterior spondylolisthesis between the two adjacent vertebrae. This is performed, prior to actuation of the locking screws, by rotating bar 5130 (and/or abutment member 532) prior to so as to forcibly displace the posterior aspect of the superior articulating processes of the inferior vertebra (along surface K) anteriorly relative to member 512 and the attached superior vertebral bone.

The device of FIG. 33 can also be used to exert a downward force onto the superior aspect of the superior articulating processes of the inferior vertebra (along surface K) distract the vertebral bones in the vertical plane. Since downward force can be excreted on either side of the midline, this maneuver can be used to correct or compensate for scoliotic forces and/or deformity. Further, the device may be positioned so that the facet joint surfaces are distracted away from one another and “off loaded” the joint by reducing the forces acting upon it. This can lead to a significant reduction in back pain (termed “Facet Joint Syndrome”) attributable to loading and movement of a disease facet joint. Finally, the device can be used to mechanically position the Functional Spinal Unit (FSU) into slight anterior flexion. This vertebral re-alignment would limit extension and enlarge the cross-sectional area of the spinal canal. Thus, the device can be used to adventurously re-align the vertebra in a number of planes while still maintain motion between them.

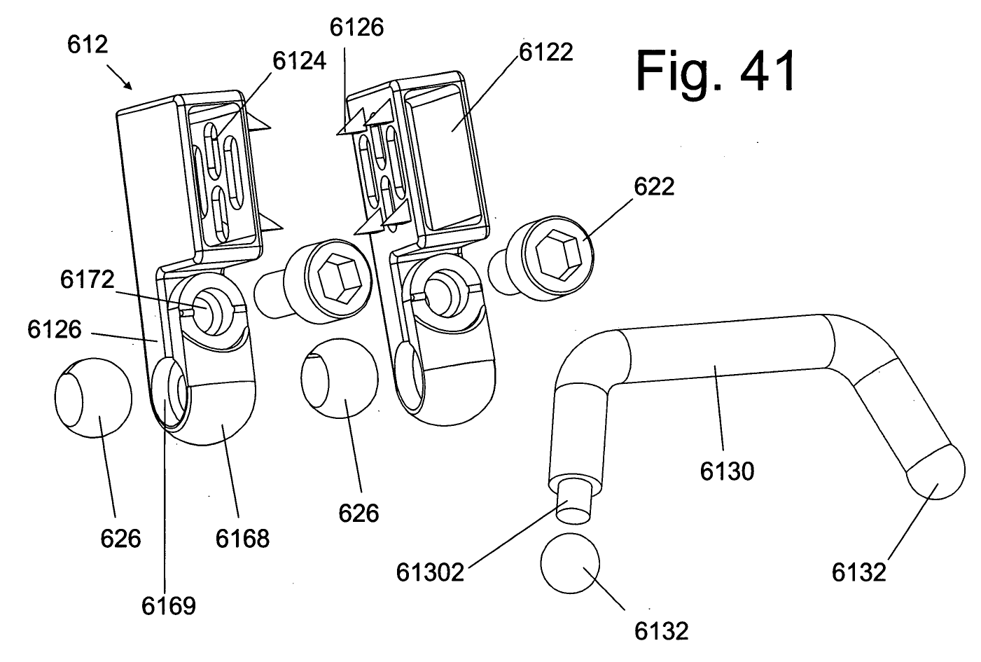

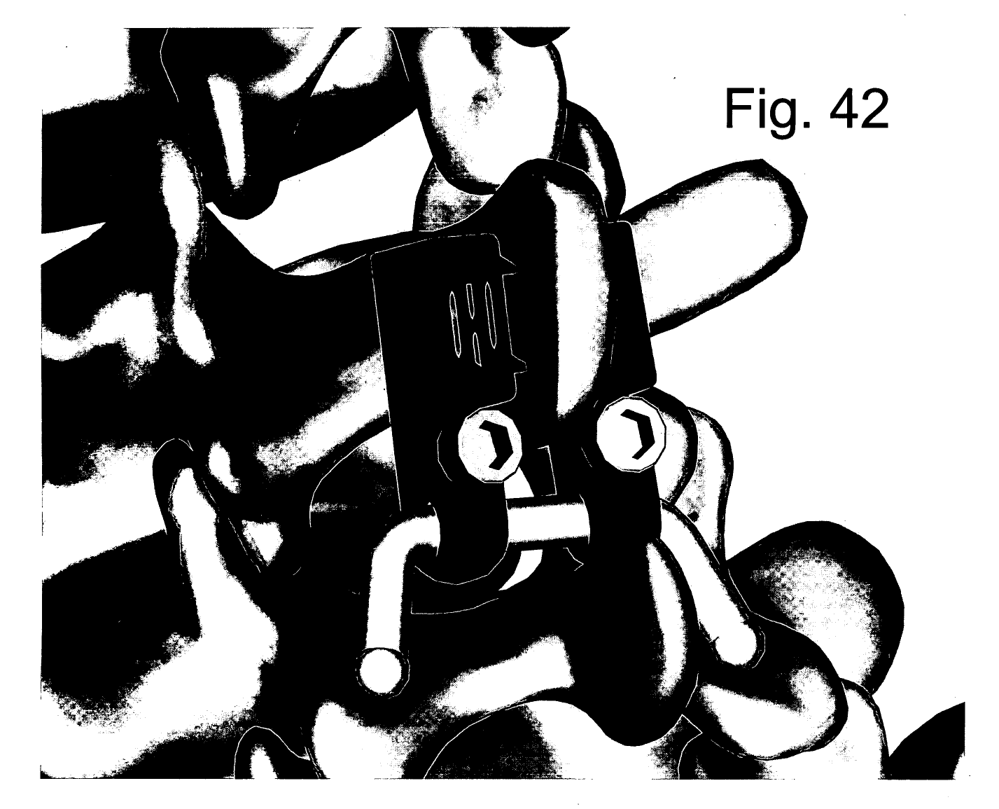

As noted, removal of any segment of the articulating processes (and/or facet joint) in not required for device implantation or mechanical manipulation of the spine. However, the operating surgeon can, if desired, supplement the procedure with nerve element decompression. FIGS. 39A and 39B show, by way of example, an embodiment of the nerve element decompression that may be employed. FIG. 39A illustrates the intact spinal segment, wherein FIG. 39B illustrates the segment 801 of bone removed from the lamina and medial aspect of the inferior articulating process of the upper vertebral bone. Segment 802 shows the segment of bone removed from the lamina and medial aspect of the superior articulating process of the lower vertebral bone. Since the inferior articulation process of the upper vertebra is anatomically positioned posterior to the superior articulation process of the lower vertebra, the total extent of resection of the superior articulation process of the lower vertebra is not fully shown in this illustration.

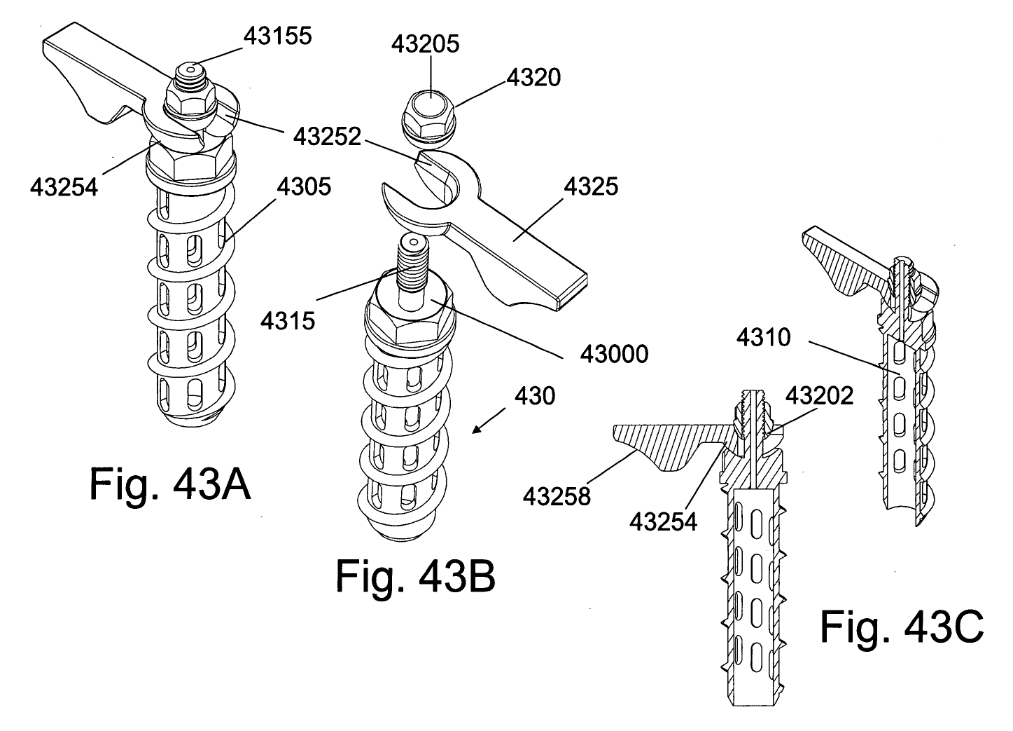

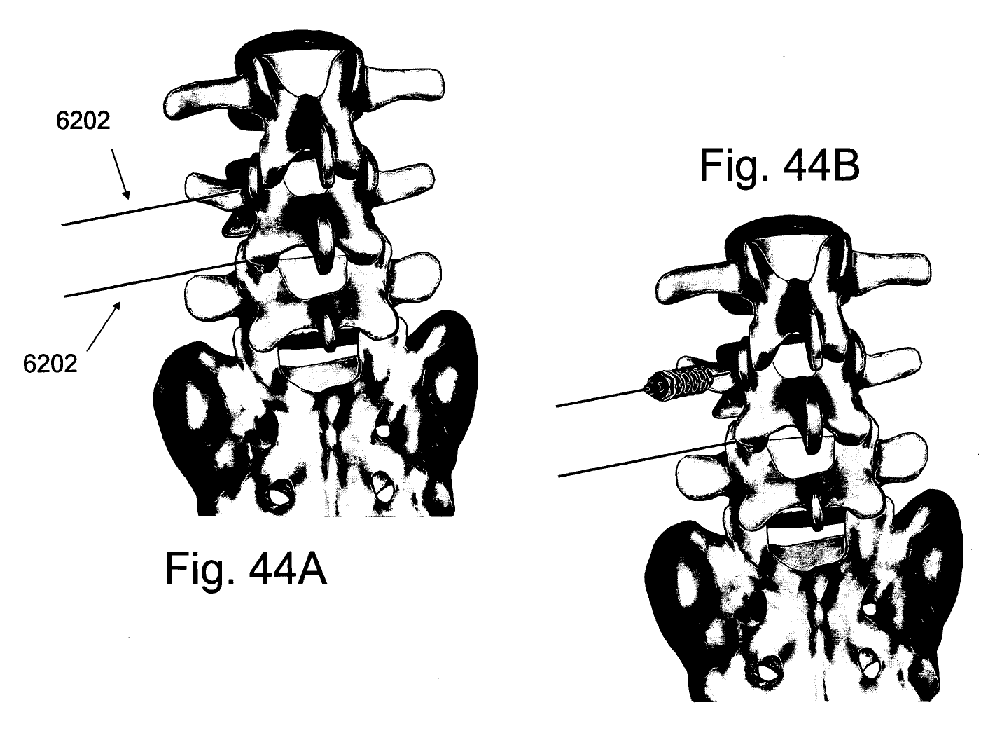

FIG. 40 shows an additional device embodiment in an assembled state. FIG. 41 shows the device of FIG. 40 in an exploded state. The device includes a pair of substantially L-shaped members 612 that are interlinked by a contoured bar 6130. Each member 612 has an internal compartment 6122 that is adapted to receive and house a bone graft or bone graft substitute. Multiple bores 6124 are contained within the medial wall that defines the compartment 6122. The bores 6124 permit communication between the bone graft material within compartment 6122 and the adjacent spinal bone, so that a bony fusion could be established between the bone graft within compartment 6122 and the adjacent spine. Multiple spiked protrusions 6126 permit device fixation to the adjacent bone. Split segment 6168 forms central bore 6169. Locking screw 622 (threads not shown) is adapted to reside within threaded bore 6172.

In the assembled state, a split locking sphere 626 resides within the central bore 6169 of member 6168. Bar 6130 resides within the central bore of split locking sphere 626. Rotation and advancement of locking screw 622 within threaded bore 6172 produces closure of split segment 6168 and reduction of the diameter of central bore 6169. The split locking sphere 626 is compressed and bar 6130 is immobilized relative to member 612. In this way the device is rigidly locked.

The bar 6130 has an end protrusion 6132 on each end, wherein the protrusions can be spherical. At least one end 6132 is removable so that the bar 6130 can be passed through the bore of locking spheres 626 during device assembly. The removable protrusion 6132 contains a threaded bore that can be threadably attached to threaded end 61302 after device assembly. In this way, the device is retained in the assembled configuration. Note that the compartment 6122 may contain bores that open onto the side bone, as depicted. As an alternative (or in addition) to the side bores, compartment 6122 may contain at least one bore on the surface that abuts, or is closest to, the lamina portion of the vertebral level to which the device is attached. The latter bore holes would permit bone growth between the fusion material inside compartment 6122 and the lamina that is adjacent (and anterior) to the device.

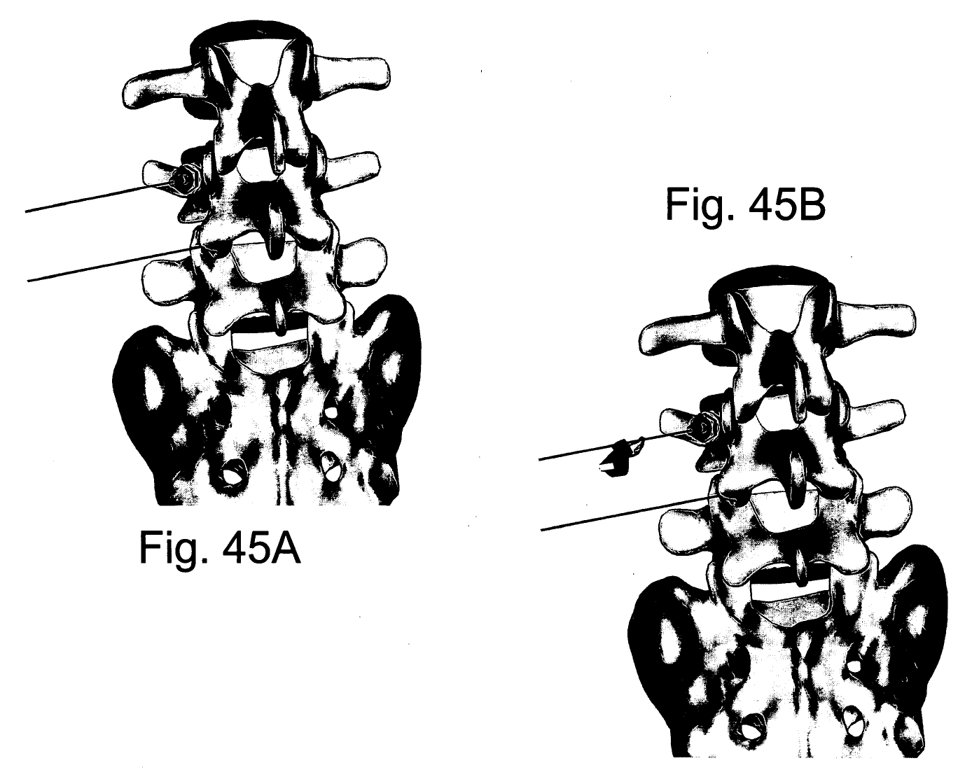

FIG. 42 shows the device of FIG. 40 attached to a spinal model. Those skilled in the art will appreciate that actual vertebral bodies include anatomical details not shown in these figures. In placement onto the vertebral bone, bar 6130 is rotated and positioned until each end protrusion 6132 abuts the posterior surface of the lamina and/or inferior articulating protrusion of a second vertebra (preferably the inferior level). Having been positioned on opposed sides of the spinous process of a first vertebra (preferably the superior level), each member 612 is then forced medially by a locking tool, such as, for example, a pair of pliers so as drive spiked protrusions 6126 into the lateral aspect of the spinous process of the first vertebra (preferably the superior level). Each locking screw 622 is then deployed to render the device rigid. Each compartment 6122 may be packed with bone-forming material before or after device attachment to bone.

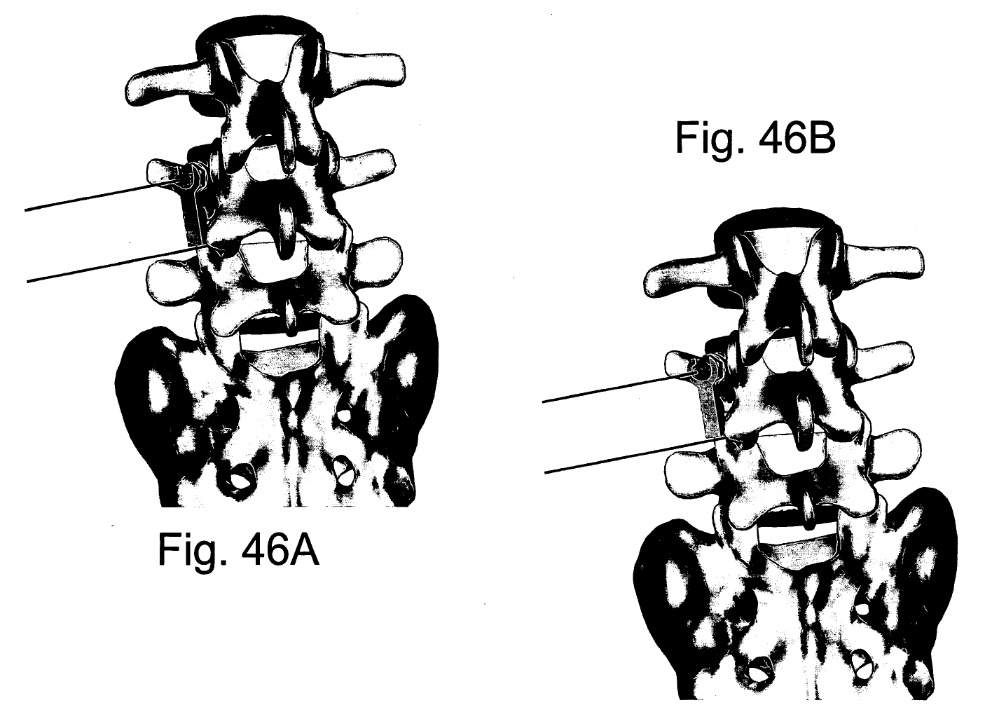

The prior embodiments disclosed devices adapted to form an additional articulation between the superior and inferior vertebra without resection and/or replacement of segments of the anatomical facet joints. In those embodiments, the implant was preferably attached and fused onto the spinous process and/or lamina of superior vertebral bone. Consequently, these implants can not be used in patients who have undergone surgical laminectomy (because the lamina and spinous process have been removed). FIG. 43 illustrate an additional embodiment wherein the device contains at least one cavity adapted to contain a bone graft material and fuse with the pedicle portion of superior vertebral bone. The device further contains an abutment surface that is adapted to abut the superior and/or posterior aspects of the superior articulation process of the lower vertebral bone (along surface K), wherein, preferably, the joint capsule of the facet joint remains substantially intact. This embodiment is also particularly adapted for percutaneous implantation and the method of implantation is disclosed.

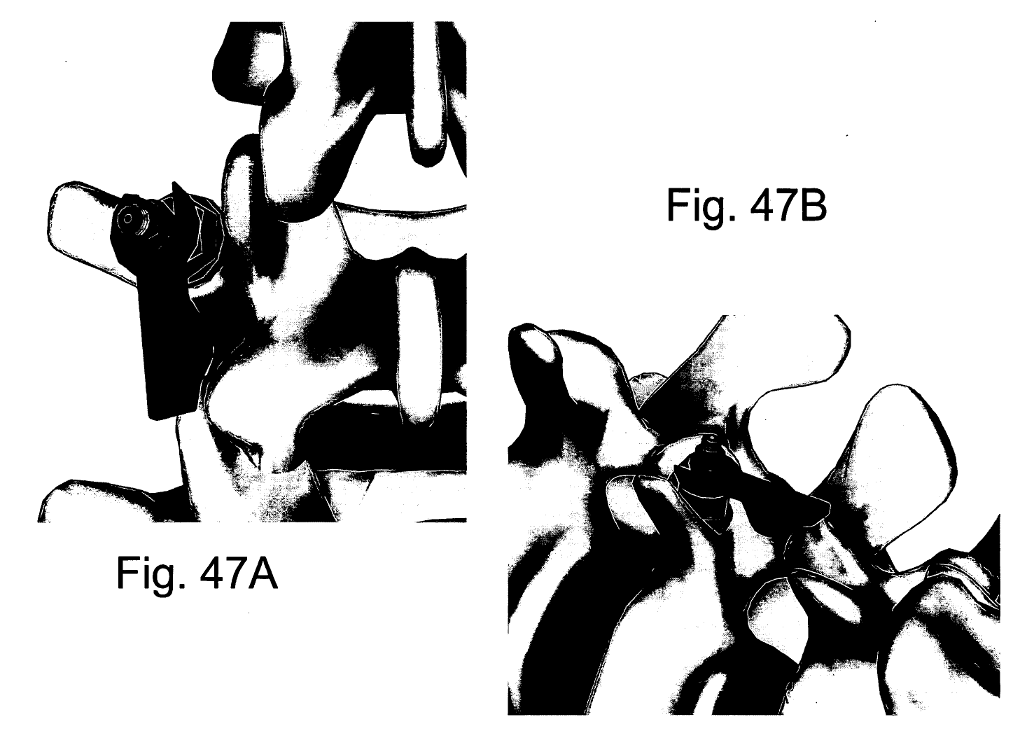

FIG. 43A illustrates the assembled device. An exploded view is shown in FIG. 43B while section views are shown in FIG. 43C. A method for the percutaneous implantation of the device under X-ray or imaging guidance is illustrated in FIGS. 44 to 47.

With reference to FIGS. 43A-43C, member 430 comprises a body that extends along a longitudinal axis. A raised helical member 4305 winds around the outer surface of the body. As shown in the cross-sectional views of FIG. 43C, the body includes an internal chamber 4310 defines by a cylindrical outer wall. A plurality of openings extend through the cylindrical outer wall. The openings permit communication between a bone graft material within internal chamber 4310 and the adjacent spinal bone, so that a bony fusion can be established between the bone graft within chamber 4310 and the adjacent spinal bone.

With reference still to FIGS. 43A-43C, a shank 4315 extends upwardly from the body. The shank 4315 has a threaded outer surface that threadbly mates with a locking nut 4320. Shank 4315 has central bore 43155 adapted to accepted pins 6202. Locking nut 4320 has a rounded bottom surface 43202 that mates with a complementary-shaped, rounded seat of a member 4325. Locking nut 4320 also has threaded bore 43205 (threads not shown). The spherical bottom of locking nut 4320 interacts with the complimentary spherical cut out 43252 of member 4325. Spherical bottom 43254 of member 4325 interacts with spherical surface 43000 of member 430. This permits member 4325 to assume a variable spatial orientation relative to member 430 and to be locked into that position by nut 4320. Member 4325 has abutment surface 43258 that is sized and shaped to abut surface K of the superior articulating process of the inferior vertebra.

FIGS. 44-47 show a method of percutaneous implantation of the device under X-ray and/or image guidance. As shown in FIG. 44A, a pair of pins 6202 are disposed on adjacent vertebral bones onto the pedicle entry point of the superior vertebra and the ipsilateral facet joint between the superior and inferior vertebrae. The pins are guided to position by x-ray and/or image guidance. The pins serve as guides for percutaneously guiding the device onto the bone. As shown in FIG. 44B, member 430 is guided to the pedicle entry point of the superior vertebra. (Prior to implantation, the cavity 4310 has been packed with bone graft material). Member 430 is rotated and threaded into the pedicle and seated as shown in FIG. 45A. In FIG. 45B, member 4325 is guided to the receiving portion of member 430 and then member 4325 is rotated till its distal tip abuts pin 6202 in the facet joint. This maneuver rotated seats abutment surface 43258 onto surface K—as shown in FIG. 46A. Locking nut 4320 is used to rigidly lock member 4325 to member 430 as shown in FIG. 46B. (An antirational feature (not shown) may be added to member 430 to prevent rotation in the pedicel). The pins are removed, leaving the implanted device as shown in FIG. 47. While illustrated on one side of the midline, a device is preferably placed on each side of the midline. The choice of the length of member 4325 will determine the extent of distraction applied between the superior and inferior vertebrae.

The disclosed devices or any of their components can be made of any biologically adaptable or compatible materials. Materials considered acceptable for biological implantation are well known and include, but are not limited to, stainless steel, titanium, tantalum, combination metallic alloys, various plastics, resins, ceramics, biologically absorbable materials and the like. Any components may be also coated/made with osteo-conductive (such as deminerized bone matrix, hydroxyapatite, and the like) and/or osteo-inductive (such as Transforming Growth Factor “TGF-B,” Platelet-Derived Growth Factor “PDGF,” Bone-Morphogenic Protein “BMP,” and the like) bio-active materials that promote bone formation. Further, any surface may be made with a porous ingrowth surface (such as titanium wire mesh, plasma-sprayed titanium, tantalum, porous CoCr, and the like), provided with a bioactive coating, made using tantalum, and/or helical rosette carbon nanotubes (or other carbon nanotube-based coating) in order to promote bone in-growth or establish a mineralized connection between the bone and the implant, and reduce the likelihood of implant loosening. Lastly, the system or any of its components can also be entirely or partially made of a shape memory material or other deformable material.

As will be apparent to those of skill in the art upon reading this disclosure, each of the individual embodiments described and illustrated herein has discrete components and features which may be readily separated from or combined with the features of any of the other several embodiments without departing from the scope of the subject matter described herein. Any recited method can be carried out in the order of events recited or in any other order which is logically possible.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

1. An orthopedic implant adapted to be implanted within a vertebral facet joint and adapted to maintain motion between adjacent vertebral bodies, comprising:
a first segment that rigidly attaches to a facet joint surface of a first vertebra, wherein the first segment contains a cavity that houses a bone forming material which forms a bony fusion with the first vertebra; and
a second segment having an abutment surface with a facet joint surface of a second vertebra, wherein the second segment does not rigidly attach to the second vertebra.

2. A device as in claim 1, wherein the device is implanted using x-ray guidance and a percutaneous technique.

3. A device as in claim 1, wherein the implanted device increases the distance between the articulation surfaces of a facet joint.

4. A device as in claim 1, wherein the implanted device at least partially limits anterior movement of the lower vertebra relative to the upper vertebra in the horizontal plane.

5. A device as in claim 1, wherein the implanted device at least partially reduces an anterior spondylolisthesis.

6. An orthopedic implant adapted to be implanted onto a vertebra segment outside of a facet joint and further adapted to maintain motion between adjacent vertebral bodies, comprising:
a first segment that rigidly attaches to a portion of an upper vertebra, wherein the segment contains a cavity that houses a bone forming material which forms a bony fusion with the upper vertebra;
a second segment forming an abutment surface with the superior articulating process of the lower vertebra, wherein the second segment is not rigidly attached to the lower vertebra.

7. A device as in claim 6, wherein the first segment of the implanted device fuses onto the spinous process portion of the upper vertebra.

8. A device as in claim 6, wherein the first segment of the implanted device fuses onto the lamina portion of the upper vertebra.

9. A device as in claim 6, wherein the first segment of the implanted device fuses onto the pedicle portion of the upper vertebra.

10. A device as in claim 6, wherein the implanted device at least partially limits anterior movement of the lower vertebra relative to the upper vertebra in the horizontal plane.

11. A device as in claim 6, wherein the implanted device can at least partially reduce an anterior spondylolisthesis.

12. A device as in claim 6, wherein the implanted device at least partially limits vertebral extension.

13. A method of maintaining motion between adjacent vertebral bodies, comprising:
implanting a device such that first a first segment of the device rigidly attaches to a facet joint surface of a first vertebra, wherein the first segment contains a cavity that houses a bone forming material which forms a bony fusion with the first vertebra and a second segment abuts a facet joint surface of a second vertebra, wherein the second segment does not rigidly attach to the second vertebra.
